(19) United States
(12) Patent Application Publication
(10) Pub. No.: US 2026/0226538 A1
(43) Pub. Date: Aug. 6, 2026

(54) POLYMERASE FOR SEQUENCING BY SYNTHESIS

(71) Applicant: 10x Genomics, Inc., Pleasanton, CA (US)

(72) Inventors: Ruijie Zhang, Milpitas, CA (US); Katherine Nakama, Dublin, CA (US); Aarushi Grover, Fremont, CA (US)

(21) Appl. No.: 19/431,036

(22) Filed: Dec. 23, 2025

Related U.S. Application Data
(60) Provisional application No. 63/740,504, filed on Dec. 31, 2024.

(51) Int. Cl. C12Q 1/6869; C12N 9/12; C12P 19/34

(52) U.S. Cl. CPC C12Q 1/6869; C12N 9/1252; C12P 19/34; C12Y 207/07007

(57) ABSTRACT

Modified polymerases capable of incorporating modified nucleotides are provided. In some aspects, the modified polymerases at least 80% identical to SEQ ID NO: 3 and comprising the following amino acid mutations: an L436 mutation selected from L436Y, L436S, and L436T; a Y437 mutation selected from Y437A and Y437G; and at least one of: a D165A mutation and a E167A mutation. In some aspects, the modified polymerases are used in a sequencing reaction, such as an in situ sequencing reaction.

### REFERENCE TO AN ELECTRONIC SEQUENCE LISTING

The contents of the electronic sequence listing (900299-459-SL.xml; Size: 13,899 bytes; and Date of Creation: Dec. 18, 2025) are herein incorporated by reference in its entirety.

### FIELD

The present disclosure relates in some aspects to modified polymerases capable of incorporating modified nucleotides. In some aspects, the modified polymerases function at temperatures at which tissues are stable. Also described are methods of using the modified polymerases to incorporate nucleotides into polynucleotides, particularly in the context of DNA sequencing and in situ DNA sequencing, as well as kits comprising components to perform said methods.

### BACKGROUND

Nucleic acid sequencing is a versatile tool that helps scientists advance the understanding of biology and has wide-ranging applications in various fields, such as medical diagnostics, biotechnology, forensic biology, and virology. For example, in situ sequencing is an advanced sequencing method that provides spatial resolution of gene expression within preserved spatial architecture of a biological sample. Reagents such as enzymes for in situ sequencing are needed.

### SUMMARY

In one aspect of the present disclosure, there is provided, a modified polymerase comprising an amino acid sequence that is at least 80% identical to SEQ ID NO: 3 and comprising the following amino acid mutations: an L436 mutation selected from L436Y, L436S, and L436T; a Y437 mutation selected from Y437A and Y437G; and at least one of: a D165A mutation and a E167A mutation.

In certain embodiments, the Y437 mutation is Y437A. In certain embodiments, the L436 mutation is L436Y. In certain embodiments, the modified polymerase comprises the D165A mutation and the E167A mutation. In certain embodiments, the modified polymerase further comprises a P438 mutation selected from: P438V, P438L, and P431I. In certain embodiments, the modified polymerase further comprises an A515L mutation. In certain embodiments, the modified polymerase has the sequence of SEQ ID NO: 1.

In certain embodiments, the modified polymerase further comprises at least one of: a Y412F mutation, a V417I mutation, a V612H mutation, an I706K mutation, and a V710M mutation. In certain embodiments, the modified polymerase further comprises: a Y412F mutation, a V417I mutation, a V612H mutation, an I706K mutation, and a V710M mutation. In certain embodiments, the polymerase has the sequence of SEQ ID NO: 2.

In certain embodiments, the modified polymerase comprises an amino acid sequence that is at least 85%, at least 90%, or at least 95% identical to SEQ ID NO: 3.

In certain embodiments, the modified polymerase is capable of incorporating modified nucleotides containing each of the four bases A, T, C and G.

In certain embodiments, the modified polymerase is capable of incorporating modified nucleotides at a reaction temperature from about 30° C. to about 80° C. In further certain embodiments, the polymerase is capable of incorporating modified nucleotides at a reaction temperature from about 40° C. to about 65° C. In still further certain embodiments, the polymerase is capable of incorporating modified nucleotides at a reaction temperature from about 45° C. to about 60° C. In still further certain embodiments, the polymerase is capable of incorporating modified nucleotides at a reaction temperature of about 60° C. In still further certain embodiments, the polymerase is capable of incorporating modified nucleotides at a reaction temperature of about 45° C.

In certain embodiments, the modified nucleotides each comprise a 3′ blocking group. In further certain embodiments, the 3′ blocking group is 3′-OR. In still further certain embodiments, the R is selected from the group consisting of: azidomethyl, allyl, methyl, methyl carbamate, hydroxymethyl, amine, ester, disulfide, sulfate, and phosphate.

In certain embodiments, at a subset of the modified nucleotides are linked to a dye. In further certain embodiments, the dye is a fluorescent dye.

In certain embodiments, the polymerase is capable of incorporating modified nucleotides in a sequencing-by-synthesis (SBS) reaction. In further certain embodiments, the polymerase is capable of incorporating modified nucleotides in an in situ sequencing-by-synthesis (SBS) reaction.

In another aspect, there is provided a method for sequencing a template nucleic acid molecule comprising:
(a) contacting a template nucleic acid molecule bound to a priming strand with: (i) a modified polymerase and (ii) a first plurality of modified nucleotide molecules comprising a 3′ blocking group, thereby incorporating a modified nucleotide molecule into the priming strand; and
(b) detecting a presence of the incorporated modified nucleotide molecule in the priming strand to identify a complementary nucleotide in the template nucleic acid molecule.

In a related aspect, there is provided a method for sequencing a template nucleic acid molecule in situ, the method comprising:
(a) providing a cell sample or tissue sample attached to a solid support, wherein the cell sample or tissue sample comprises a template nucleic acid molecule bound to a priming strand,
(b) contacting the template nucleic acid molecule bound to the priming strand with (i) a modified polymerase as provided herein and (ii) a first plurality of modified nucleotide molecules comprising a 3′ blocking group, thereby incorporating a modified nucleotide into the priming strand; and
(c) detecting a presence of the incorporated modified nucleotide molecule in the priming strand to identify a complementary nucleotide in the template nucleic acid molecule.

In certain embodiments, the first plurality of modified nucleotide molecules includes:
(i) modified nucleotide molecules of a first nucleobase type, each attached to a first dye,
(ii) modified nucleotide molecules of a second nucleobase type, each attached to a second dye,
(iii) modified nucleotide molecules of a third nucleobase type, each attached to a third dye, and
(iv) modified nucleotide molecules of a fourth nucleobase type.

In certain further embodiments, the modified nucleotide molecules of the fourth nucleobase are each attached to a fourth dye type. In certain other further embodiments, the modified nucleotide molecules of the fourth nucleobase type are each not attached to a dye. In still further certain embodiments, the four nucleobase types comprise:
(i) an adenine or an analogue of adenine as the first nucleobase type,
(ii) a cytosine or an analogue of cytosine as the second nucleobase type,
(iii) a guanine or an analogue of guanine as the third nucleobase type, and
(iv) a thymine or an analogue of thymine or a uracil or an analogue of uracil as the fourth nucleobase type.

In certain embodiments, the 3′ blocking group comprises O-azidomethyl, O-allyl, O-methyl, O-methyl carbamate, O-hydroxymethyl, O-amine, O-ester, O-disulfide, O-sulfate, and/or O-phosphate.

In certain embodiments, the method further comprises the additional steps of:
d) unblocking the reversible blocking group, and
e) contacting the priming strand bound to the template nucleic acid molecule with a modified polymerase and a second plurality of modified nucleotide molecules each comprising a 3′ blocking group, thereby incorporating a nucleotide molecule of the second plurality of nucleotide molecules into the priming strand.

In certain further embodiments, the unblocking of the reversible blocking group comprises exposing the reversible blocking group to a reducing agent. In still further certain embodiments, the reducing agent is dithiothreitol (DTT), tris-2-carboxyethylphosphine hydrochloride (TCEP), sodium borohydride, hydrogen peroxide, and/or formic acid.

In certain embodiments, the method further comprises repeating a cycle of steps (a)-(b) of the method and unblocking the reversible blocking group by exposing the reversible blocking group to a reducing agent for at least one additional cycle, thereby identifying an additional complementary nucleotide in the template nucleic acid molecule. In still further certain embodiments, the method further comprises repeating the additional cycle for at least 2, 5, 10, 20, 30, 40, 50, 75, 100, 125, or 150 additional cycles.

In certain embodiments, the modified nucleotide of the first plurality comprises a dye attached to the modified nucleotide by a linker moiety, wherein the linker moiety comprises a cleavable moiety, and the method further comprises: cleaving the cleavable moiety to release the dye. In further certain embodiments, the cleaving comprises contacting the cleavable moiety with a reducing agent. In still further certain embodiments, the cleavable moiety comprises an O-azidomethyl group.

In certain embodiments, the template nucleic acid molecule comprises DNA. In certain embodiments, the template nucleic acid molecule comprises a rolling circle amplification product. In further certain embodiments, the rolling circle amplification product is amplified from a circularized probe that hybridizes to a target nucleic acid molecule. In further certain embodiments, the circularizable probe or probe set is a padlock probe. In still further certain embodiments, the circularizable probe, wherein, upon hybridizing, is hybridized to two regions of the target nucleic acid molecule separated by a gap, and the method further comprises, prior to ligating, polymerizing a 3′ end of the circularizable probe to fill the gap. In still further certain embodiments, the target nucleic acid molecule is an mRNA molecule and the polymerizing is catalyzed by a reverse transcriptase. In still further certain embodiments, the gap is between 2 to 40 nucleotides in length.

In certain embodiments, the target nucleic acid molecule is an mRNA molecule. In certain embodiments, the target nucleic acid molecule is a DNA molecule. In still further certain embodiments, the gap is between 2 to 40 nucleotides in length.

In certain embodiments, the template nucleic acid molecule comprises a barcode sequence associated with a target analyte. In certain further embodiments, the method further comprises prior to step (a) of the method, hybridizing a circularizable probe or probe set to the target analyte or to a labeling agent bound to the target analyte and ligating the circularizable probe or probe set to form a circularized probe, wherein the method further comprises performing rolling circle amplification of the circularized probe to generate the template nucleic acid molecule.

In certain embodiments, the template nucleic acid molecule to be sequenced is attached to a solid support.

In certain embodiments, the target nucleic acid molecule comprises a nucleotide variation, a nucleotide polymorphism, a mutation, a substitution, an insertion, a deletion, a translocation, a rearrangement, a duplication, an inversion, and/or a repetitive sequence

In certain embodiments, the target nucleic acid molecule comprises a sequence of an immune molecule. In certain further embodiments, the target nucleic acid molecule comprises an antigen receptor transcript. In still further certain embodiments, the antigen receptor transcript is a T cell receptor (TCR) transcript, optionally wherein the TCR transcript comprises a TCRα VJ join, a TCRβ VDJ join, a TCRγ VJ join, or a TCRδ VDJ join. In still further certain other embodiments, the antigen receptor transcript is an immunoglobulin (Ig) transcript, optionally wherein the Ig transcript comprises an Igκ VJ join, an Igλ VJ join, or an IgH VDJ join.

In certain embodiments, the method comprises identifying multiple different antigen receptor transcripts present at a plurality of locations in the biological sample. In certain further embodiments, the multiple different antigen receptor transcripts present at a plurality of locations in the biological sample comprise a plurality of VDJ joins of the multiple different antigen receptor transcripts comprising at least about 50, at least about 100, at least about 500, at least about 1,000, at least about 5,000, at least about 10,000, or more VDJ joins of different sequences.

In certain embodiments, the template nucleic acid molecule comprises a sequence of a perturbation agent introduced to the cell sample or tissue sample before step (a). In certain further embodiments, the template nucleic acid molecule comprises a spacer sequence of a perturbation agent. In certain further other embodiments, the template nucleic acid molecule comprises a unique barcode specific to a guide RNA. In certain further other embodiments, the template nucleic acid molecule comprises a barcode sequence.

In certain embodiments, the template nucleic acid molecule comprises a CRISPR molecule, a nucleic acid molecule edited using the CRISPR molecule, and/or a precursor or derivative thereof.

In another aspect, there is provided a method for sequencing a template nucleic acid molecule in situ comprising:
(a) providing a cell sample or tissue sample attached to a solid support, wherein the cell sample or tissue sample comprises a template nucleic acid molecule bound to a priming strand,
(b) contacting the template nucleic acid molecule bound to the priming strand with (i) a modified polymerase as provided herein and (ii) a first plurality of modified nucleotide molecules comprising a 3′ blocking group, thereby incorporating a modified nucleotide into the priming strand; and
(c) detecting a presence of the incorporated modified nucleotide molecule in the priming strand to identify a complementary nucleotide in the template nucleic acid molecule.

In certain embodiments, the first plurality of modified nucleotide molecules includes:
(i) modified nucleotide molecules of a first nucleobase type, each attached to a first dye,
(ii) modified nucleotide molecules of a second nucleobase type, each attached to a second dye,
(iii) modified nucleotide molecules of a third nucleobase type, each attached to a third dye, and
(iv) modified nucleotide molecules of a fourth nucleobase type.

In certain further embodiments, the modified nucleotide molecules of the fourth nucleobase are each attached to a fourth dye type. In certain other further embodiments, the modified nucleotide molecules of the fourth nucleobase type are each not attached to a dye. In still further certain embodiments, the four nucleobase types comprise:
(i) an adenine or an analogue of adenine as the first nucleobase type,
(ii) a cytosine or an analogue of cytosine as the second nucleobase type,
(iii) a guanine or an analogue of guanine as the third nucleobase type, and
(iv) a thymine or an analogue of thymine or a uracil or an analogue of uracil as the fourth nucleobase type.

In certain embodiments, the 3′ blocking group comprises O-azidomethyl, O-allyl, O-methyl, O-methyl carbamate, O-hydroxymethyl, O-amine, O-ester, O-disulfide, O-sulfate, and/or 0-phosphate.

In certain embodiments, the method further comprises the additional steps of:
d) unblocking the reversible blocking group, and
e) contacting the priming strand bound to the template nucleic acid molecule with a modified polymerase and a second plurality of modified nucleotide molecules each comprising a 3′ blocking group, thereby incorporating a nucleotide molecule of the second plurality of nucleotide molecules into the priming strand.

In certain further embodiments, the unblocking of the reversible blocking group comprises exposing the reversible blocking group to a reducing agent. In still further certain embodiments, the reducing agent is dithiothreitol (DTT), tris-2-carboxyethylphosphine hydrochloride (TCEP), sodium borohydride, hydrogen peroxide, and/or formic acid.

In certain embodiments, the method further comprises repeating a cycle of steps (a)-(b) of the method and unblocking the reversible blocking group by exposing the reversible blocking group to a reducing agent for at least one additional cycle, thereby identifying an additional complementary nucleotide in the template nucleic acid molecule. In still further certain embodiments, the method further comprises repeating the additional cycle for at least 2, 5, 10, 20, 30, 40, 50, 75, 100, 125, or 150 additional cycles.

In certain embodiments, the modified nucleotide of the first plurality comprises a dye attached to the modified nucleotide by a linker moiety, wherein the linker moiety comprises a cleavable moiety, and the method further comprises: cleaving the cleavable moiety to release the dye. In further certain embodiments, the cleaving comprises contacting the cleavable moiety with a reducing agent. In still further certain embodiments, the cleavable moiety comprises an O-azidomethyl group.

In certain embodiments, the template nucleic acid molecule comprises DNA. In certain embodiments, the template nucleic acid molecule comprises a rolling circle amplification product. In further certain embodiments, the rolling circle amplification product is amplified from a circularized probe that hybridizes to a target nucleic acid molecule. In further certain embodiments, the circularizable probe or probe set is a padlock probe. In still further certain embodiments, the circularizable probe, wherein, upon hybridizing, is hybridized to two regions of the target nucleic acid molecule separated by a gap, and the method further comprises, prior to ligating, polymerizing a 3′ end of the circularizable probe to fill the gap. In still further certain embodiments, the target nucleic acid molecule is an mRNA molecule and the polymerizing is catalyzed by a reverse transcriptase. In still further certain embodiments, the gap is between 2 to 40 nucleotides in length.

In certain embodiments, the target nucleic acid molecule is an mRNA molecule. In certain embodiments, the target nucleic acid molecule is a DNA molecule. In still further certain embodiments, the gap is between 2 to 40 nucleotides in length.

In certain embodiments, the template nucleic acid molecule comprises a barcode sequence associated with a target analyte. In certain further embodiments, the method further comprises prior to step (a) of the method, hybridizing a circularizable probe or probe set to the target analyte or to a labeling agent bound to the target analyte and ligating the circularizable probe or probe set to form a circularized probe, wherein the method further comprises performing rolling circle amplification of the circularized probe to generate the template nucleic acid molecule.

In certain embodiments, the template nucleic acid molecule to be sequenced is attached to a solid support.

In certain embodiments, the tissue sample is a tissue section. In certain further embodiments, the tissue sample is a formalin-fixed, paraffin-embedded (FFPE) sample, a frozen tissue sample, or a fresh tissue sample.

In certain embodiments, the cell sample comprises a layer of cells deposited on a surface.

In certain embodiments, the cell sample or tissue sample is fixed and/or permeabilized.

In certain embodiments, the cell sample or tissue sample is crosslinked and/or embedded in a matrix, optionally wherein the matrix comprises a hydrogel.

In certain embodiments, the biological sample is cleared.

In certain embodiments, the target nucleic acid molecule comprises a nucleotide variation, a nucleotide polymorphism, a mutation, a substitution, an insertion, a deletion, a translocation, a rearrangement, a duplication, an inversion, and/or a repetitive sequence

In certain embodiments, the target nucleic acid molecule comprises a sequence of an immune molecule. In certain further embodiments, the target nucleic acid molecule comprises an antigen receptor transcript. In still further certain embodiments, the antigen receptor transcript is a T cell receptor (TCR) transcript, optionally wherein the TCR transcript comprises a TCRα VJ join, a TCRβ VDJ join, a TCRγ VJ join, or a TCRδ VDJ join. In still further certain other embodiments, the antigen receptor transcript is an immunoglobulin (Ig) transcript, optionally wherein the Ig transcript comprises an Igκ VJ join, an Igλ VJ join, or an IgH VDJ join.

In certain embodiments, the method comprises identifying multiple different antigen receptor transcripts present at a plurality of locations in the biological sample. In certain further embodiments, the multiple different antigen receptor transcripts present at a plurality of locations in the biological sample comprise a plurality of VDJ joins of the multiple different antigen receptor transcripts comprising at least about 50, at least about 100, at least about 500, at least about 1,000, at least about 5,000, at least about 10,000, or more VDJ joins of different sequences.

In certain embodiments, the template nucleic acid molecule comprises a sequence of a perturbation agent introduced to the cell sample or tissue sample before step (a). In certain further embodiments, the template nucleic acid molecule comprises a spacer sequence of a perturbation agent. In certain further other embodiments, the template nucleic acid molecule comprises a unique barcode specific to a guide RNA. In certain further other embodiments, the template nucleic acid molecule comprises a barcode sequence.

In certain embodiments, the template nucleic acid molecule comprises a CRISPR molecule, a nucleic acid molecule edited using the CRISPR molecule, and/or a precursor or derivative thereof.

In another aspect, there is provided a kit for sequencing a template nucleic acid molecule comprising: a plurality of modified nucleotide molecules each comprising a 3′ blocking group, and a modified polymerase.

In certain embodiments, the kit further comprises a primer designed to hybridize to a template nucleic acid molecule and/or one or more probes that are designed to hybridized to a target analyte, optionally wherein the one or more probes are circularizable probes.

In certain embodiments, the kit further comprises one or more additional components for in situ sequencing of a target analyte in a cell sample or tissue sample. In certain further embodiments, the one or more additional components comprises one or more: imaging slides, cell staining reagents, nucleic acid stains, membrane stains (e.g., cellular or nuclear membrane), cytological stains, immunohistochemical reagents, or combinations thereof.

### BRIEF DESCRIPTION OF THE DRAWINGS

The drawings illustrate certain features and advantages of this disclosure. These embodiments are not intended to limit the scope of the appended claims in any manner.

FIG. 1 shows a phylogeny and percent identity matrix for wild type polymerases “CR43”; “RX72”; “9HY46”; and two previously characterized polymerases: 9° N and Vent.

FIG. 2 shows the design of single nucleotide incorporation assays as used herein.

FIG. 3 shows results of a single nucleotide incorporation assay at different polymerase reaction temperatures, calculated as percent incorporation of a modified 3′ blocked cytosine nucleotide following incubation with four different engineered polymerases at 45° C., or 60° C.

FIG. 4 shows results of a single nucleotide incorporation assay at different polymerase reaction temperatures, calculated as percent incorporation of modified nucleotides, cytosine-dye #1 and guanine-dye # following incubation with 9YH46 mutant 2 or RX72, or a control polymerase at 45° C., or 50° C.

### DETAILED DESCRIPTION

Embodiments of the present disclosure are based in pertinent part on generation of new modified polymerases that are capable of incorporating 3′ modified nucleotides. The polymerases disclosed herein may have particular advantages at low concentrations. The enzymes disclosed herein are particularly useful for in situ sequencing and kits for the same.

The modified polymerases are capable of incorporating a modified nucleotide molecule, including at temperatures of 45° C. and 60° C. (see Example 3).

### 1. Polymerases

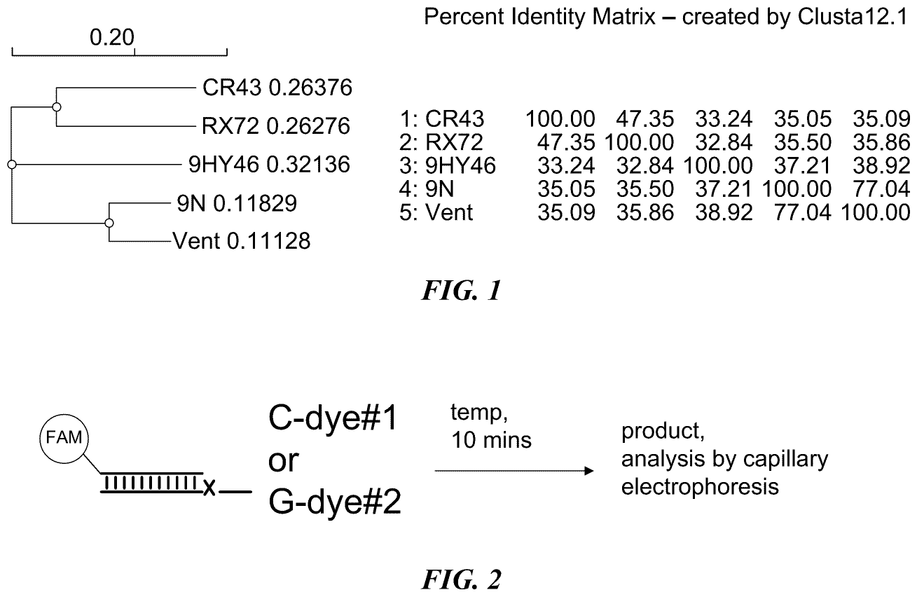

The present disclosure provides modified polymerases. In some embodiments, the modified polymerase is a family B type DNA polymerase, or a mutant or variant thereof. Family B polymerases are enzymes involved in DNA replication and repair, found in bacteria, archaea, and eukaryotes. Examples of Family B DNA polymerases include E. coli DNA Polymerase II (Pol II) and I (Pol I), eukaryotic DNA Polymerases 6 (Pol 6) and F (Pol F), as well as 9° N polymerase, Thermococcus kodakaraenis (KOD) polymerase, and Thermococcus litoralis (Vent) polymerase e, all of which are involved in DNA synthesis 9° N polymerase, KOD polymerase, Vent polymerase are thermostable Family B polymerases that have previously been mutated to improve incorporation of 3′ modified nucleotides, for example, for sequencing-by-synthesis. As shown in FIG. 1, the polymerase of SEQ ID NO: 3 (a sequence of a putative polymerase of Thermodesulfovibrio aggregans) shares only 37% sequence identity with 9° N wild-type polymerase (SEQ ID NO: 4) and 39% identity with Vent wild-type polymerase. In contrast, wild-type Vent polymerase shares about 77% sequence identity with 9° N wild-type polymerase. Thus, modified polymerases provided herein (including 9HY46 mutant 1 and 9HY46 mutant 2) are designed using a vastly different backbone than known thermostable Family B enzymes and the ability of these modified polymerases to incorporate 3′ modified nucleotide molecules in a sequencing reaction would not have been expected based on known family B polymerases.

In certain preferred embodiments, the modified polymerase is engineered from a Thermodesulfovibrio aggregans polymerase.

In certain embodiments, the modified polymerase has an amino acid sequence that is at least 80% identical to SEQ ID NO: 3, and comprises at least one exonuclease inactivating amino acid mutation, and at least one amino acid mutation that improves incorporation of modified nucleotide molecules. In some embodiments, the at least one amino acid mutation that improves incorporation of modified nucleotide molecules is selected from: a L436 mutation, a Y437 mutation, a P438 mutation, and a A515 mutation. In some embodiments, the at least one exonuclease inactivating amino acid mutation is selected from: a D165 mutation and an E167 mutation. In some embodiments, the at least one exonuclease inactivating amino acid mutation is a D165 mutation and an E167 mutation. In some embodiments, the D165 mutation is selected from D165A and D165G. In some embodiments, the E167 mutation is selected from E167A and E167G. In certain embodiments, the modified polymerase has an amino acid sequence that is at least 80% identical to SEQ ID NO: 3, and comprises at least one exonuclease inactivating amino acid mutation, and at least one of the following amino acid mutations: (i) a Y437 mutation selected from Y437A and Y437G, (ii) a L436 mutation selected from L436Y, L436S, and L436T.

In certain embodiments, the modified polymerase comprises an amino acid sequence that is at least 80% identical to SEQ ID NO: 3 and comprises the following amino acid mutations: (i) a Y437 mutation selected from Y437A and Y437G, (ii) a L436 mutation selected from L436Y, L436S, and L436T, and (iii) and at least one of: a D165A mutation and a E167A mutation. In certain embodiments, the Y437 mutation is Y437A. In certain embodiments, the L436 mutation is L436Y. In certain embodiments, the modified polymerase comprises a D165A mutation and a E167 mutation. In certain embodiments, the modified polymerase further comprises a P438 mutation, a A515L mutation, or a P438 mutation and a A515L mutation. In certain embodiments, the P438 mutation is selected from: P438V, P438L, and P438I. In certain embodiments, the modified polymerase has the amino acid sequence of SEQ ID NO: 3 and the following mutations: D165A, E167A, Y437A, and P438V. In certain preferred embodiments, the modified polymerase has the amino acid sequence of SEQ ID NO: 3 and the following mutations: D165A, E167A, L436Y, Y437A, P438V, and A515L. In certain embodiments, the modified polymerase comprises the following mutations: D165A, E167A, L436I, Y437A, P438V, and A515L. In certain embodiments, the modified polymerase is at least 95%, at least 96%, at least 97%, at least 98%, or at least 99% identical to SEQ ID NO: 1. In certain embodiments, the modified polymerase comprises SEQ ID NO: 1.

In certain embodiments, the modified polymerase comprises an amino acid sequence that is at least 85%, at least 90%, or at least 95% identical to SEQ ID NO: 3.

In certain embodiments, the modified polymerase is at least 80% identical to SEQ ID NO: 3, comprises the following amino acid mutations: (i) a Y437 mutation selected from Y437A and Y437G, (ii) a L436 mutation selected from L436Y, L436S, and L436T, and (iii) and at least one of: a D165A mutation and an E167A mutation, and further comprises at least one of: a Y412F mutation, a V417I mutation, a V612H mutation, an I706K mutation, and a V710M mutation. In certain embodiments, the modified polymerase further comprises: a Y412F mutation, a V417I mutation, a V612H mutation, an I706K mutation, and a V710M mutation. In some embodiments, the modified polymerase comprises the following mutations: D165A, E167A, L436I, Y437A, A515E, Y412F, V417I, V612H, I706K, and V710M. In certain embodiments, the modified polymerase is at least 95%, at least 96%, at least 97%, at least 98%, or at least 99% identical to SEQ ID NO: 2. In certain preferred embodiments, the modified polymerase comprises SEQ ID NO: 2.

Differences between a reference polynucleotide or polypeptide and a variant thereof may be determined by methods routinely practiced in the art to determine identity, which are designed to give the greatest match between the sequences tested. Methods to determine sequence identity can be applied from publicly available computer programs. Computer program methods to determine identity between two sequences include, for example, BLASTP, BLASTN (Altschul, S. F. et al., J. Mol. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al., NCBI NLM NIH Bethesda, MD).

In certain embodiments, the modified polymerase is capable of incorporating modified nucleotides at a reaction temperature from about 30° C. to about 80° C. In certain preferred embodiments, the modified polymerase is capable of incorporating modified nucleotides at a reaction temperature from about 40° C. to about 65° C. In certain embodiments, the modified polymerase is capable of incorporating modified nucleotides at a reaction temperature from about 45° C. to about 60° C. In certain embodiments, the modified polymerase is capable of incorporating modified nucleotides at about 60° C. or about 45° C.

In some embodiments, the modified polymerase is capable of incorporating modified nucleotides containing each of the four bases, adenine (A), cytosine (C), guanine (G), thymine (T), and uracil (U). In some embodiments, the modified nucleotides each comprises a 3′ blocking group. In certain preferred embodiments, the 3′ blocking group is 3′-OR and R is selected from the group consisting of: azidomethyl, allyl, methyl, methyl carbamate, hydroxymethyl, amine, ester, disulfide, sulfate, and phosphate. In certain preferred embodiments at least a subset of the modified nucleotides are linked to a dye.

As used herein, the term “incorporation” refers to joining of the modified nucleotide to the free 3′ hydroxyl group of a second nucleotide via formation of a phosphodiester linkage with the 5′ phosphate group of the modified nucleotide. The second nucleotide to which the modified nucleotide is joined will typically occur at the 3′ end of a polynucleotide chain.

As used herein, the term “modified polymerase” refers to a polymerase that has at least one amino acid change compared to a control polymerase enzyme. In some embodiments, the amino acid change is the substitution of at least one amino acid for another amino acid. Said substitution may include conservative and/or non-conservative substitutions. In some embodiments, the amino acid change may comprise a deletion or addition of one or more amino acids from or to the protein, provided that the polymerase is able to incorporate modified nucleotides.

Examples of other DNA polymerases known in the art include Taq polymerase (a heat-stable polymerase from Thermus aquaticus), 9° N-7 DNA polymerase (or variants thereof, for example, D141A/E143A/A485L), phi29 polymerase (has high processivity and fidelity and low strand displacement activity; derived from φ29, a phage of Bacillus subtilis), Klenow fragment (an exonuclease deficient fragment of DNA polymerase I), Bacillus stearothermophilus DNA polymerase (BST), T4 DNA polymerase, T7 DNA polymerase, and DNA polymerase I.

In some embodiments, the modified polymerase is an “isolated” or “purified” polypeptide. The term “isolated polypeptide” refers to a polypeptide that is essentially free from contaminating cellular components, such as carbohydrates, lipids, nucleic acids or other proteinaceous impurities which may be associated with the polypeptide in nature. Typically, a preparation of the isolated polymerase contains the polymerase in a highly purified form, e.g., at least about 80% pure, at least about 90% pure, at least about 95% pure, at least about 98% pure, and at least about 99% pure. Methods of polypeptide purification are known in the art, including, but not limited to affinity chromatography, ion exchange chromatography, size exclusion chromatography, precipitation (e.g., by ammonium sulfate, and ultra-filtration (Scopes, R. K. (1994). Protein Purification: Principles, High-Resolution Methods, and Applications. New York: Springer). Purity of a preparation of the enzyme may be assessed, for example, by the presence of a single band in Sodium dodecyl sulfate polyacrylamide gel electrophoresis (SDS-PAGE) gel analysis.

In some embodiments, the polymerase is a DNA polymerase, the nucleotide molecules include deoxyribonucleotide molecules, and the template comprises DNA. In some embodiments, the template comprises cDNA.

As used here, the term “comparator polymerase” or “control polymerase” refers to as the polymerase against which the activity of the modified polymerase is compared. In one embodiment of the invention, the control polymerase may comprise a “wild-type” 9° N DNA polymerase or a variant thereof comprising exonuclease inactivating mutations and mutations that increase incorporation of modified nucleotide molecules. Unless otherwise stated, “wild-type” refers to a polymerase comprising its natural amino acid sequence, as it would be found in nature. The comparator polymerase can also comprise any known polymerase, including mutant polymerases known in the art.

### 2. Sequencing System Components

The present disclosure provides one or more components for a sequencing reaction, including in situ sequencing reactions. In some embodiments, the components for sequencing include one or more of: polymerase molecules, nucleotide molecules, and primer molecules.

### Polymerase Molecules

In some embodiments, the one or more components for sequencing, including in situ sequencing, include polymerase molecules, e.g., a modified polymerase, as described in section I of the Detailed Description above.

### Nucleotide Molecules

In some embodiments, the one or more components for sequencing, including in situ sequencing, include a plurality of nucleotide molecules. The nucleotide molecule may be any nucleotide molecules capable of selectively base pairing with a complementary nucleotide in a template strand to be sequence. Nucleotide molecules include a sugar and a nucleobase. In some embodiments, the sugar of the nucleotide molecule is selected from ribose, 2′ deoxyribose, and 1′,2′ dideoxyribose. In particular embodiments, the sugar is 2′ deoxyribose. In some embodiments, the nucleobase for each of the plurality of nucleotide molecules is selected from adenine (A), cytosine (C), guanine (G), thymine (T), and uracil (U).

In some embodiments, the one or more components for sequencing, including in situ sequencing, include a plurality of modified nucleotide molecules each comprising a 3′ blocking group. In some embodiments, the 3′ blocking group is 3′-OR. In some embodiments, the R is selected from the group consisting of: azidomethyl, allyl, methyl, methyl carbamate, hydroxymethyl, amine, ester, disulfide, sulfate and phosphate. In particular embodiments, the 3′ blocking group is O-azidomethyl.

In some embodiments, nucleotide molecules provided herein comprise one or more detectable labels. The detectable labels may be distinguishable by means of their differences in fluorescence, Raman spectrum, charge, mass, refractive index, luminescence, length, or any other measurable property.

Detectable labels can be suitable for small scale detection and/or suitable for high-throughput screening. As such, suitable detectable labels include, but are not limited to, radioisotopes, fluorophores, chemiluminescent compounds, bioluminescent compounds, quantum dots, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some instances, the detectable label is bound to another moiety, for example, a nucleotide or nucleotide analog, and can include a fluorescent, a colorimetric, or a chemiluminescent label.

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected.

In some instances the detectable label is a dye. Dyes used as detectable labels are capable of absorbing and/or emitting light at specific desired wavelengths. In some instances, the dye is a fluorescent dye. Fluorescent dyes are capable of absorbing and emitting light at specific wavelengths. Examples of molecules that can act as fluorescent dyes include coumarin or coumarin-derivative dye, cyanine or a cyanine-derivative dye, fluorescein or a fluorescein-derivative dye, rhodamine or a rhodamine-derivative dye, or phenoxazine or a phenoxazine-derivative dye. Examples of cyanine or cyanine-derivative dyes include Cyanine 2 (Cy2), Cyanine 5 (Cy5), and Cyanine 3 (Cy3). Examples of rhodamine-derivative dyes include Rhod-2, Rhodamine B, RHODAMINE GREEN™, RHODAMINE RED™, Rhodamine Phalloidin, Rhodamine 110, Rhodamine 123, and 5-ROX (carboxy-X-rhodamine). Further examples of fluorescent dyes include 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-532, ATTO-643, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, CALCIUM CRIMSON™, CALCIUM GREEN-1™, CALCIUM ORANGE™, Calcofluor® White, 5-Carboxyfluoroscein (5-FAM), 5-Carboxynaphthofluoroscein, 6-Carboxyrhodamine 6G, 5-Carboxytetramethyirhodamine (5-TAMRA), Carboxy-X-rhodamine (5-ROX), Cascade Blue®, CASCADE YELLOW™, CCF2 (GENEBLAZER™), CFP (Cyan Fluorescent Protein), CFP/YFP FRET, Chromomycin A3, Cl-NERF (low pH), CPM, 6-CR 6G, CTC Formazan, CY2®, CY3®, CY3.5®, CY5®, CY5.5®, CY7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FLUORX®, FLUORO-GOLD™ (high pH), FLUORO-GOLD™ (low pH), Fluoro-Jade, FM® 1-43, Fura-2 (high calcium), Fura-2/BCECF, FURA RED™ (high calcium), FURA RED™/Fluo-3, GENEBLAZER™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LYSOSENSOR™ Blue (pH 5), LYSOSENSOR™ Green (pH 5), LYSOSENSOR™ Yellow/Blue (pH 4.2), LysoTracker® Green, LysoTracker® Red, LysoTracker® Yellow, Mag-Fura-2, Mag-Indo-1, MAGNESIUM GREEN™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, RHODAMINE GREEN™, RHODAMINE RED™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), SODIUM GREEN™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some instances, the dye is a fluorescent dye for example as described, for example, in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

In some aspects, the nucleotide molecules include nucleotide molecules attached to a detectable label via a linker moiety. In some embodiments, a nucleotide molecule of a nucleotide molecule as described herein may be attached to a linker moiety. In some embodiments, a linker includes a moiety generated by conjugating a nucleotide molecule to the detectable label.

In some embodiments, the linker is polar and/or charged. In some embodiments, the linker is not non-polar. In some embodiments, the linker is polar. Examples of polar moieties that may be included in the linker include poly(ethylene oxide), poly(propylene oxide), carbamate, ester aldehydes, ketones, and succinimide groups such as thiosuccinimide. In some embodiments, the linker includes one or more poly(ethylene oxide) moieties. In some embodiments, the linker includes one to five poly(ethylene oxide) moieties.

In some embodiments, a linker includes a moiety generated by click chemistry conjugation of a first click chemistry reacting group attached to a nucleotide molecule with a second click chemistry group attached to the detectable moiety. In some instances, the nucleotide molecule and the linker are conjugated via first and second click reactive functional groups using a click reaction. In some embodiments, the first click reactive functional group and second click reactive functional group are selected from: azido/alkynyl groups; alkynyl/azido groups; azido/dibenzocyclooctynyl (DBCO) groups; dibenzocyclooctynyl (DBCO)/azido groups; azido/cyclooctynyl groups; cyclooctynyl/azido groups; tetrazine/dienophile groups; dienophile/tetrazine groups; thiol/alkynyl groups; alkynyl/thiol groups; cyano/1,2-amino thiol groups; 1,2-amino thiol/cyano groups; nitrone/cyclooctynyl groups; cyclooctynyl/nitrone groups; or any combination thereof.

In some instances, the linker includes a cleavable linker. In some instances, the cleavable linker comprises a photocleavable linker, a Pd-cleavable linker, or a reducing agent-cleavable linker such as a phosphine-cleavable linker or a disulfide linker.

In some instances, the cleavable linker includes a photocleavable linker. Any suitable photocleavable linker can be used (see, e.g., Seo et al. (2005), PNAS 102(17): 5926-5931, which is incorporated by reference herein in its entirety). In some instances, the photocleavable linker comprises a nitrobenzyl group. For instance, a photocleavable nitrobenzyl linker can be cleaved using laser irradiation (e.g., 355 nm, 10 seconds, 1.5 Wcm-2).

In some instances, the cleavable linker includes a Pd-cleavable linker. Any suitable Pd-cleavable linker can be used (see, e.g., Ju et al. (2006), PNAS 103(52): 19635-19640, which is incorporated by reference herein in its entirety). In some instances, the Pd-cleavable linker comprises an allyl group. For instance, a Pd-cleavable allyl linker can be cleaved using incubation with a Na₂PdCl₄/P(PhSO₃Na)₃ mixture (e.g., 30 seconds at 70° C.).

In some instances, the cleavable linker is a reducing agent-cleavable linker. Examples of reducing agents that may be used to cleave a reducible linker include Tris(2-carboxyethyl) phosphine and dithiothreitol (DTT). Examples of reducible moieties that may be included in a linker include disulfide and azidomethyl. In some instances, the cleavable linker includes a phosphine-cleavable linker. Any suitable phosphine-cleavable linker can be used (see, e.g., Guo et al. (2008), PNAS 105(27): 9145-9150, which is incorporated by reference herein in its entirety). In some instances, the phosphine-cleavable linker comprises an azido group. For instance, a phosphine-cleavable azido linker can be cleaved using incubation with a Tris(2-carboxyethyl) phosphine (TCEP) mixture (e.g., 15 minutes at 65° C.).

In some instances, the cleavable linker includes a disulfide bond. For instance, the disulfide bond can be cleaved using incubation with a reducing agent, such as beta-mercaptoethanol, TCEP, or dithiothreitol (DTT).

### Sequencing Primer

In some embodiments, the one or more components for in situ sequencing include a sequencing primer. A sequencing primer may be designed to anneal to a template nucleic acid molecule directly upstream of a portion to be sequenced. In some embodiments, for in situ sequencing of a panel of a plurality of different template nucleic acid sequences, at least two, at least three, or at least four different sequencing primers are used.

A primer is generally a single-stranded nucleic acid sequence having a 3′ end that can be used as a substrate for a nucleic acid polymerase in a nucleic acid extension reaction. The sequence of nucleotides added during the extension process is determined by the sequence of the template polynucleotide. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer may in some cases refer to a primer binding sequence.

### 3. Sequencing Methods and In Situ Sequencing Methods

The disclosed sequencing components may be used in any nucleic acid-based assays, including those performed in cell or tissue samples, such as in situ sequencing of a cell or tissue sample attached to a solid support.

In some embodiments, the sequencing method includes sequencing a template nucleic acid molecule by:
a) contacting a template nucleic acid molecule bound to a priming strand with: (i) a polymerase as provided herein and (ii) a first plurality of modified nucleotide molecules comprising a 3′ blocking group, thereby incorporating a modified nucleotide molecule into the priming strand; and
b) detecting a presence of the incorporated modified nucleotide molecule in the priming strand to identify a complementary nucleotide in the template nucleic acid molecule.

In some embodiments, the nucleotide molecule includes a cleavage moiety between the nucleotide molecule and the dye, and the method further comprises cleaving the cleavage moiety to release the dye, thereby releasing the dye from the nucleotide molecule.

In some embodiments, the sequencing method includes sequencing a template nucleic acid molecule in situ.

In some embodiments, the contacting the template nucleic acid molecule bound to the priming strand with the modified polymerase and the first plurality of modified nucleotide molecules comprising a 3′ blocking group is performed at a temperature from about 30° C. to about 80° C., preferably from about 40° C. to about 65° C., such as from about 40° C. to about 45° C., about 45° C. to about 50° C., about 50° C. to about 55° C., about 55° C. to about 60° C., about 40° C. to about 50° C., about 50° C. to about 60° C., or about 45° C. to about 60° C.

In some instances, the disclosed methods may further comprise processing optical signals (e.g., fluorescence signals) detected in images (e.g., fluorescence images) acquired during the cyclic series of base-by-base sequencing reactions to detect the presence or absence of complementary detectably labeled nucleotides in each sequencing cycle at the locations of each of a plurality of template nucleic acid molecules (e.g., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences). In some instances the detection step may comprise the use of an optical imaging technique (e.g., a fluorescence imaging technique) and real time or post-processing measurement of optical signals (e.g., fluorescence signals or the absence thereof) associated with the presence of a specific nucleotide molecule covalently coupled to the modified 3′ blocked nucleotide of the priming strand at a plurality of locations corresponding to a plurality of target analytes distributed throughout the biological sample or tethered to specific locations on a substrate surface (e.g., a flow cell surface).

In some instances, the cyclic series of base-by-base sequencing reactions includes at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 60, 70, 80, 90, 100, 125, 150 or more than 150 cycles of the base-by-base sequencing reaction. In some instances, the cyclic series of base-by-base sequencing reactions includes 1 to 150 cycles, such as 5 to 125, 10 to 100, and 20 to 80 cycles, of the base-by-base sequencing reaction.

Sequencing reactions as described herein may include sequencing-by-synthesis, whereby the nucleotide is incorporated into a priming strand, or sequencing-by-binding, wherein the nucleotide is transiently bound to a polymerase and priming strand during detection. In some embodiments, the nucleotide molecule includes a cleavable linker between the nucleotide molecule and the dye, and the linker is cleaved following incorporation and detection of the detectable label.

In some embodiments, the sequencing reaction is a sequencing-by-synthesis reaction. In some embodiments, upon contacting each priming strand bound to a template nucleic acid molecule with a polymerase and a nucleotide, the nucleotide becomes incorporated into an extended priming strand.

In some instances, the sequencing reaction is a sequencing-by-binding reaction. In some embodiments, the priming strand is blocked from incorporation of the nucleotide molecule. In some embodiments, the first wash conditions are configured to not disrupt the bound nucleotide/polymerase/priming strand complex. In some instances, the first wash step may comprise, for example, use of the same buffer used for contacting the primed template nucleic acid with a polymerase and nucleotide molecules (but without the polymerase and nucleotide molecules). In some instances, the first wash buffer may not include KCl and/or may include little to no DMSO. In some instances, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some embodiments, each cycle of base-by-base sequencing-by-binding using the nucleotide molecules described herein further includes a second wash step following the detection step in order to disrupt the complex. In some instances, the second wash is performed under more stringent conditions than the first wash. For example, the second wash may include a temperature higher than room temperature (e.g., 30-40° C.), a higher salt concentration (e.g., a higher KCl salt concentrations (e.g., at least 50 mM KCl)), a solvent miscible in the wash buffer solution (e.g., dimethyl sulfoxide (DMSO)), a detergent (e.g., sodium dodecyl sulfate (SDS)), or a combination thereof.

In some instances, the (non-covalently bound) complex consisting of the 3′ terminus of the priming strand, the template nucleic acid molecule, the polymerase, and a nucleotide molecule may comprise a transient complex. In some instances, the transient complex may persist for at least 5 sec, 10 sec, 20 sec, 30 sec, 40 sec, 50 sec, 1 min, 2 min, 3 min, 4 min, 5 min, or 10 min after removal of polymerase and nucleotides used to contact the primed template nucleic acid molecule and form the complex. The “persistence time” of the complex, as used herein, refers to the average length of time that the complex remains stable without significant dissociation of any of the components of the bound complex.

In some instances, the sequencing reaction comprises repeating one or more sequencing cycles. In some instances, the sequencing reaction comprises repeating the same sequencing cycle.

In some instances, a sequencing cycle of the sequencing includes contacting the template with the polymerase and a plurality of nucleotide molecules including first nucleotide molecules having a first base type and a first dye, second nucleotide molecules having a second base type and a second dye, third nucleotide molecules having a third base type and a third dye, and fourth nucleotide molecules having a fourth base type, wherein the first, second, and third dyes are each different. In some instances, the fourth nucleotide molecules are fourth nucleotide molecules. In some instances, the fourth nucleotide molecules are not linked to a dye. In some embodiments, the fourth nucleotide molecules are linked to a fourth dye that is different from the first, second, and third dyes.

In some embodiments, a sequencing cycle comprises contacting one or more templates with the polymerase and a mixture of nucleotide molecules comprising: (i) first nucleotide molecules having a first base type and a first dye; (ii) second nucleotide molecules having a second base type and a second dye; (iii) third nucleotide molecules having a third base type and a third dye; and (iv) fourth nucleotide molecules having a fourth base type and a fourth dye, wherein the first, second, third, and fourth bases types are different (e.g., A, T/U, C, and G) and the first, second, third, and fourth dyes are different (e.g., each detectable in a different color channel using fluorescence microscopy).

In some embodiments, a sequencing cycle comprises contacting one or more templates with the polymerase a mixture of nucleotide molecules comprising: (i) first nucleotide molecules having a first base type and a first dye; (ii) second nucleotide molecules having a second base type and a second dye; (iii) third nucleotide molecules having a third base type and a third dye; and (iv) fourth nucleotide molecules having a fourth base type, wherein the first, second, third, and fourth bases types are different (e.g., A, T/U, C, and G) and the first, second, and third dyes are different (e.g., each detectable in a different color channel using fluorescence microscopy). In some embodiments, the fourth nucleotide molecules are not detectably labeled or are detectably labeled but are not detected.

In some embodiments, a sequencing cycle comprises contacting one or more templates with the polymerase and a mixture of nucleotide molecules comprising: (i) first nucleotide molecules each having the same first base type and being dual labeled or configured to be dual labeled with a first dye and a second dye (e.g., for the first base type, approximately half of nucleotides of the first base type have a first dye and approximately half have a second dye); (ii) second nucleotide molecules each having the same second base type and the first dye (but no second dye); (iii) third nucleotide molecules each having the same third base type and the second dye (but no first dye); and (iv) fourth nucleotide molecules each having the same fourth base type and no first dye or second dye, wherein the first, second, third, and fourth bases types are different (e.g., A, T/U, C, and G) and the first and second dyes are different (e.g., each detectable in a different color channel using fluorescence microscopy).

In some embodiments, the sequencing reaction involves a repeating pattern of a sequencing cycle comprising two separate steps: a first step and a second step. In some embodiments, the first step comprises contacting one or more templates with the polymerase a mixture of nucleotide molecules comprising: (i) first nucleotide molecules each having the same first base type and a dye linked to the first nucleotide molecule, where the linkage is cleavable; (ii) second nucleotide molecules each having the same second base type and the dye linked to the second nucleotide molecule, where the linkage is not cleavable or cleavable but not cleaved in the second step; (iii) third nucleotide molecules each having the same third base type and a binding moiety configured to bind to the dye but is not bound to the dye in the first step; and (iv) fourth nucleotide molecules each having the same fourth base type and no dye linked thereto and no binding moiety configured to bind to the dye, wherein the first, second, third, and fourth bases types are different (e.g., A, T/U, C, and G). In some embodiments, the second step comprises: cleaving the dyes linked to the first nucleotide molecules, wherein the linkage between the dyes and the second nucleotide molecules is not cleavable or cleavable but not cleaved; contacting the third nucleotide molecules with the dye to allow it to bind to the binding moiety, thereby labeling the third nucleotide molecules.

In some instances, the first plurality of nucleotides (or one or more additional pluralities of nucleotide molecules) are selected from modified A, T, U, C, and G. In some instances, the first plurality of nucleotides (or one or more additional pluralities of nucleotide molecules) are selected from modified A, T, C, and G.

In some instances, the first plurality of nucleotide molecules or at least one additional plurality of nucleotide molecules may each comprise nucleotide molecules that do not include a 3′ reversible terminator moiety.

In some instances, the first plurality of nucleotide molecules and at least one additional plurality of nucleotide molecules may each comprise at least one nucleotide molecule that is not labeled with a detectable label.

Methods for processing the series of optical signals detected over the course of performing a cyclic series of base-by-base sequencing reactions to identify a nucleotide sequence are described elsewhere herein.

The polymerases described in Section I of the present Detailed Description may be used for performing the disclosed methods.

In some embodiments, the priming strand comprises a 3′ terminal nucleotide that is reversibly blocked. Thus, the nucleotide does not become incorporated into the priming strand during formation of the complex. In some embodiments, the method further comprises the additional steps of: disrupting the complex, unblocking the reversibly blocked 3′ terminal nucleotide molecule of the priming strand, and contacting the priming strand bound to the template nucleic acid molecule with a polymerase and a second plurality of nucleotide molecules, thereby incorporating a nucleotide molecule of the second plurality of nucleotide molecules into the priming strand. In some embodiments, the method further includes repeating a cycle of steps (a) contacting and (b) detecting, and the additional steps of disrupting the complex for at least one additional cycle, thereby identifying an additional complementary nucleotide in the template nucleic acid molecule. In some embodiments, the method includes repeating the cycle for at least 2, 5, 10, 20, 30, 40, 50, 60, 70, 80, 90, 100, 110, 120, 130, 140, 150 or more than 150 additional cycles. In some instances, the method includes repeating the cycle for 1 to 150 additional cycles, such as 5 to 125, 10 to 100, and 20 to 80 additional cycles.

In some embodiments, the priming strand comprises a 3′ terminal nucleotide that is unblocked, and wherein step (a) further comprises incorporating the nucleotide into the priming strand. In some embodiments, the nucleotide comprises a reversibly blocked 3′ position, and the method further comprises, after the incorporating, unblocking the reversibly blocked 3′ position. In some embodiments, the linker comprises a cleavable moiety, and the method further comprises: (c) cleaving the cleavable moiety to release the dye from the nucleotide molecule. In some embodiments, the cleavable moiety is photocleavable, and the cleaving comprises exposing the nucleotide to UV light, or the cleavable moiety is a disulfide or an O-azido moiety, and the cleaving comprises contacting the nucleotide with a reducing agent. In some embodiments, the method further includes repeating a cycle steps (a)-(c), thereby incorporating an additional nucleotide into the priming strand and identifying an additional complementary nucleotide in the template nucleic acid molecule. In some embodiments, the repeating is for at least 2, at least 5, at least 10, at least 20, or at least 30 additional cycles.

In some embodiments of any of the sequencing methods provided herein, the template nucleic acid molecule comprises a DNA molecule. In some embodiments, the template nucleic acid molecule comprises an RNA molecule, optionally wherein the RNA molecule is an mRNA molecule. In some embodiments, the template nucleic acid molecule comprises a target analyte nucleic acid molecule. In some embodiments, the template nucleic acid molecule comprises a barcode sequence associated with a target analyte.

In some embodiments, the sequencing methods further include hybridizing a circularizable probe or probe set to the target analyte or to a labeling agent bound to the target analyte and ligating the circularizable probe or probe set to form a circularized probe, wherein the method further comprises performing rolling circle amplification of the circularized probe to generate the template nucleic acid molecule. In some embodiments, the circularizable probe or probe set is a padlock probe. In some embodiments, the template nucleic acid molecule to be sequenced is attached to a solid support. In some embodiments, the template nucleic acid molecule is sequenced in situ in a cell sample or tissue sample. In some embodiments, the cell sample comprises a layer of cells deposited on a surface.

In some aspects, provided are sequencing methods using nucleotide molecules as described herein. The sequencing methods include multi-cycle sequencing approaches where a cyclic series of steps are performed to identify nucleotides base-by-base in a template nucleic acid sequence (e.g., a target analyte sequence and/or an associated target-specific barcode sequence).

In some instances, the template nucleic acid molecule includes a target analyte nucleic acid molecule (e.g., a DNA molecule, an RNA molecule, or an mRNA molecule). In some instances, the template nucleic acid includes a reporter oligonucleotide, such as a barcode.

In some instances, the template nucleic acid molecule is a DNA molecule. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA molecules can be copies from another nucleic acid molecule (e.g., DNA or RNA such as mRNA).

In some instances, the methods includes contacting a template nucleic acid molecule with a sequencing primer designed to hybridize to a portion of the template nucleic acid molecule, where the sequencing primer includes a reversibly terminated nucleotide at its 3′ end. In such embodiments, the modified, 3′ reversibly terminated nucleotide blocks incorporation of nucleotide molecules into the sugar-phosphate Such sequencing primers may be used in a sequencing-by-binding approach using the nucleotide molecules described herein.

In some instances, the methods includes contacting a template nucleic acid molecule with a sequencing primer designed to hybridize to a portion of the template nucleic acid molecule, where the sequencing primer does not include a reversibly terminated nucleotide at its 3′ end. In such embodiments, a nucleotide molecule is incorporated into the sugar-phosphate of the priming strand. Such sequencing primers may be used in a sequencing-by-synthesis approach using the nucleotide molecules described herein.

In some embodiments utilizing any of the nucleotide molecules described herein, the 3′ terminal nucleotide of the priming strand is blocked, and the method further includes the additional steps of: disrupting the complex, unblocking the reversibly blocked 3′ terminal nucleotide molecule of the priming strand, and contacting the priming strand bound to the template nucleic acid molecule with a polymerase and a second plurality of nucleotide molecules, thereby incorporating a nucleotide molecule of the second plurality of nucleotide molecules into the priming strand. In some embodiments, the method further includes repeating a cycle of steps (a) and (b) and the additional steps for at least one additional cycle, thereby identifying an additional complementary nucleotide in the template nucleic acid molecule. In some embodiments, the method includes repeating the cycle for at least 2, 5, 10, 20, or 30 additional cycles.

In some embodiments utilizing a nucleotide molecule comprising a cleavable moiety between the nucleotide molecule and the dye, the priming strand comprises a 3′ terminal nucleotide that is unblocked, and wherein step (a) further comprises incorporating the nucleotide into the priming strand. In some embodiments, the nucleotide comprises a reversibly blocked 3′ position, and the method further comprises, after the incorporating, unblocking the reversibly blocked 3′ position. In some embodiments, the linker comprises a cleavable moiety, and the method further comprises: (c) cleaving the cleavable moiety to release the dye. In some embodiments, the cleavable moiety is photocleavable and the cleaving comprises exposing the nucleotide to UV light, or the cleavable moiety is disulfide or an O-azido moiety and the cleaving comprises contacting the nucleotide with a reducing agent. In some embodiments, the sequencing cycle, thereby incorporating an additional nucleotide into the priming strand and identifying an additional complementary nucleotide in the template nucleic acid molecule. In some embodiments, the method includes repeating the cycle of steps (a)-(c) for at least 2, 5, 10, 20, or 30 additional cycles.

### Biological Samples

In some aspects, provided herein are sequencing methods for sequencing a template nucleic acid molecule in or derived from a biological sample. In some embodiments, the template nucleic acid molecule to be sequenced is attached to a solid support. In some embodiments, a template nucleic acid molecule is sequenced in situ in a cell sample or tissue sample. In some embodiments, the cell or tissue sample is attached to a solid support. In some embodiments, the cell sample includes a layer of cells deposited on a surface. In some embodiments, the tissue sample is an formalin fixed paraffin embedded tissue sample processed for in situ sequencing. In some embodiments, the tissue sample is fresh frozen tissue sample processed for in situ sequencing.

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA. In particular embodiments, the analyte includes RNA. In particular embodiments, the analyte includes RNA and/or DNA, and the sequencing method includes determining the presence of a single nucleotide polymorphism.

In some embodiments, an analyte herein is endogenous to a biological sample and can include nucleic acid analytes and non-nucleic acid analytes. Methods and compositions disclosed herein can be used to analyze nucleic acid analytes.

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some aspects, provided herein are methods of sequencing nucleic acids obtained from a biological sample. A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1 μm) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some instances, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some instances, the biological sample is prepared using formalin-fixation and paraffin-embedding (FFPE), which are established methods. In some instances, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some instances, the methods provided herein include one or more post-fixing (also referred to as post-fixation) steps. In some instances, one or more post-fixing step is performed after contacting a sample with a polynucleotide disclosed herein, e.g., one or more probes such as a circular or padlock probe. In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein includes de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, TRITON X-100™ or TWEEN-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some instances, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### Embedding

In some instances, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix may comprise a network of natural molecules and/or synthetic molecules that are chemically and/or enzymatically linked, e.g., by crosslinking. In some instances, a 3D matrix may comprise a synthetic polymer. In some instances, a 3D matrix comprises a hydrogel.

In some aspects, a biological sample is embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some instances, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some instances, one or more of the polynucleotide probe(s) and/or amplification product (e.g., amplicon) thereof can be modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

In some instances, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

To facilitate visualization, biological samples can be stained using a wide variety of stains and staining techniques. In some instances, for example, a sample can be stained using any number of stains and/or immunohistochemical reagents. One or more staining steps may be performed to prepare or process a biological sample for an assay described herein or may be performed during and/or after an assay. In some instances, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample can be segmented using one or more images taken of the stained sample.

In some instances, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples can be destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a DARPin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### Probe Hybridization

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

In some aspects, sequencing methods provided herein (e.g., in situ sequencing methods) include a ligation step. Ligation are usually carried out enzymatically to form a phosphodiester linkage between a 5′ terminal nucleotide with a 3′ terminal nucleotide.

In some embodiments, a ligation product of an endogenous analyte and/or a labeling agent is analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, sequencing methods included herein include use of a probe or probe set capable of DNA-templated ligation, such as from a cDNA molecule. See, e.g., U.S. Pat. No. 8,551,710, which is hereby incorporated by reference in its entirety. In some instances, sequencing methods included herein include use of a probe or probe set capable of RNA-templated ligation. See, e.g., U.S. Pat. Pub. 2020/0224244 which is hereby incorporated by reference in its entirety. In some instances, the probe set is a SNAIL probe set. See, e.g., U.S. Pat. Pub. 20190055594, which is hereby incorporated by reference in its entirety. In some instances, provided herein is a multiplexed proximity ligation assay. See, e.g., U.S. Pat. Pub. 20140194311 which is hereby incorporated by reference in its entirety. In some instances, sequencing methods included herein include use of a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some instances, a circular probe is indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76(14): 5584-5597, incorporated by reference herein in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves an enzymatic ligation. In some instances, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, AMPLIGASE™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some instances, the sequencing methods provided herein including analyzing a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents).

A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:10113-119, 2000; Faruqi et al, BMC Genomics 2:4, 2000; Nallur et al, Nucl. Acids Res. 29:e118, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some instances, the polymerase is phi29 DNA polymerase.

In some aspects, during the amplification step, nucleotides can be added to the reaction to incorporate the nucleotides in the amplification product (e.g., nanoball). Non-limiting examples of the nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination.

### Fluorescence Detection and Imaging

As previously described, provided herein are methods of sequencing that include detecting nucleotide molecules labelled with a dye moiety, such as a fluorophore. Methods of using the nucleotide molecules includes detection of the dye to detect the presence of the nucleotide molecule, such as for in situ sequencing of a cell or tissue sample. Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, methods disclosed herein provide surprisingly reduced tissue autofluorescence.

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some instances, non-limiting examples of techniques and methods applicable to the provided embodiments comprise those described in, for example, U.S. Pat. Nos. 4,757,141, 5,151,507 and 5,091,519.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a more simple set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method does not comprise embedding the sample or molecules from the sample in an exogenous matrix.

In some cases, analysis is performed on one or more images captured, and may comprise processing the image(s) and/or quantifying signals observed. In some instances, images of signals from different fluorescent channels and/or nucleotide incorporation cycles can be compared and analyzed. In some instances, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) is quantified and analyzed.

In some instances, sequencing methods disclosed herein (e.g., in situ sequencing methods) include sequencing a barcode sequence. Analytes described herein can be associated with one or more barcode(s), e.g., at least two, three, four, five, six, seven, eight, nine, ten, or more barcodes. Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding instances, the methods provided herein can include analyzing the barcodes performing in situ sequencing using the nucleotide molecules as disclosed herein.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4^{N} unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously.

Nucleic acid amplification may be performed using any of a variety of nucleic acid amplification techniques known to those of skill in the art, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies—A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5(4): 245-252, and U.S. Pat. No. 8,143,008, the entire contents of each of which are incorporated herein by reference. In some embodiments, the amplification reaction is a rolling circle amplification

In some instances, the disclosed methods for performing nucleic acid sequencing (e.g., in situ and/or flow cell sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences). In some instances, a primer sequence may be designed to hybridize to a probe sequence (e.g., a sequence present in a padlock probe). In some embodiments, a plurality of target sequences comprising a first subset of target sequences and a second subset of target sequences are sequenced simultaneously, and the first subset of target sequences are sequenced using a first primer sequence, and the second subset of target sequences are sequenced using a second primer sequence. Using multiple different primer sequences may be useful, for example, to reduce optical crowding.

In some instances, the disclosed methods for performing nucleic acid sequencing (e.g., in situ and/or flow cell sequencing) may comprise performing one or more steps of nucleic acid amplification or replication using one or more polymerases. Examples of polymerases that may be used for amplification include, but are not limited to, DNA polymerases (e.g., Taq DNA polymerase), RNA polymerases, and/or reverse transcriptases.

As noted elsewhere herein, non-limiting examples of polymerases for use in rolling circle amplification (RCA) comprise DNA polymerases such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some aspects, the polymerase is phi29 DNA polymerase.

As noted elsewhere herein, the disclosed methods for performing nucleic acid sequencing (e.g., in situ sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497). The characterization of errors associated with the sequencing process and implementation of chemistry-, imaging-, and/or signal processing software-based methods for minimizing sequence errors are thus important for maximizing the accuracy of sequencing results.

In four-color sequencing methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface or the location of each target analyte, or amplified representation thereof (e.g., an RCP) in the case of in situ sequencing, where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5):786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed sequencing methods may comprise sequence reads of at least about 5, 10, 20, 30, 40, 50, 60, 70, 80, 90, 100 or more nucleotides or base pairs of the template nucleic acid sequences.

In some instances, the disclosed sequencing methods may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, or more than 10⁶, sequencing reads per run.

In some instances, the disclosed sequencing methods include assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed sequencing methods include identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed sequencing methods include alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs known to those of skill in the art may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5):455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14):1838-1844), and String Graph Assembler (SGA) (see, e.g., Simpson et al. (2012), “Efficient de novo Assembly of Large Genomes Using Compressed Data Structures”, Genome Res. 22: 549-556).

In some instances, the sequencing methods described herein (e.g., in situ sequence sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., nucleotide molecules, primers, detectable-labeled probes and/or non-labeled probes, polymerases and/or other enzymes, deprotection reagents, buffers, etc.) to the biological sample (e.g., to a sample cartridge within which the biological sample is contained) and/or to remove spent reagents therefrom. In some instances, one or more sample preparation steps (e.g., fixing, embedding, and/or sample clearing) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more nucleotide mixtures (e.g., mixtures of nucleotide molecules, as well as primers, polymerases, deprotection reagents, etc.) to sequence, e.g., native nucleic acid sequences, barcode sequences associated with target analytes, or amplified copies thereof (e.g., barcode sequences included in RCP(s)) in the biological sample.

Additionally, the optics module is configured to illuminate the biological sample with light having one or more spectral emission curves (over a range of wavelengths) and subsequently capture one or more images of emitted light signals from the biological sample during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface may be processed in real time and/or at a later time to determine the sequence of the one or more nucleic acid sequences (e.g., barcode sequences associated with one or more target molecules) that have been extracted from a biological sample. In some embodiment, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges). In some instances, the sample module includes an X-Y stage configured to move the biological sample along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

In various instances, the sample analyzed is a biological sample (e.g., a tissue) that includes molecules such as DNA, RNA, proteins, antibodies, etc. For example, the sample can be a sectioned tissue that is treated to access the RNA thereof for probe (e.g., circularizable probe) hybridization and sequencing (e.g., using a sequencing primer that hybridizes to RCPs to sequence barcode sequences in the RCPs) described elsewhere herein.

In various instances, the sample is placed in the opto-fluidic instrument or system for analysis and detection of the molecules in the sample. In various instances, the opto-fluidic instrument or system is configured to facilitate the experimental conditions conducive for the detection of the target molecules. For example, the opto-fluidic instrument or system can include a fluidics module, an optics module, a sample module, and an ancillary module, and these modules may be operated by a system controller to create the experimental conditions for base-by-base sequencing of nucleic acid molecules in the sample, as well as to facilitate the imaging of the sample (e.g., by an imaging system of the optics module). In various instances, the various modules of the opto-fluidic instrument or system include components in communication with each other, or at least some of them may be integrated together.

In various instances, the sample module is configured to receive the sample into the opto-fluidic instrument or system. For instance, the sample module may include a sample interface module (SIM) that is configured to receive a sample device (e.g., cassette) onto which the sample can be deposited. That is, the sample may be placed in the opto-fluidic instrument or system by depositing the sample (e.g., the sectioned tissue) on a sample device that is then inserted into the SIM of the sample module. In some instances, the sample module may also include an X-Y stage onto which the SIM is mounted. The X-Y stage may be configured to move the SIM mounted thereon (e.g., and as such the sample device containing the sample inserted therein) in perpendicular directions along the two-dimensional (2D) plane of the opto-fluidic instrument or system.

The experimental conditions that are conducive for the detection of the molecules in the sample may depend on the target molecule detection technique that is employed by the opto-fluidic instrument or system. For example, in various instances, the opto-fluidic instrument or system can be a system that is configured to detect molecules (e.g., nucleotides incorporated into extending sequencing primers using an identifier sequence as a template) in the sample.

In various instances, the fluidics module may include one or more components that may be used for storing the reagents, as well as for transporting said reagents to and from the sample device containing the sample. For example, the fluidics module may include reservoirs configured to store the reagents, as well as a waste container configured for collecting the reagents (e.g., and other waste) after use by the opto-fluidic instrument or system to analyze and detect the molecules of the sample. Further, the fluidics module may also include pumps, tubes, pipettes, etc., that are configured to facilitate the transport of the reagent to the sample device (e.g., and as such the sample). For instance, the fluidics module may include pumps (“reagent pumps”) that are configured to pump washing/stripping reagents to the sample device for use in washing/stripping the sample (e.g., as well as other washing functions such as washing an objective lens of the imaging system of the optics module).

In various instances, the ancillary module can be a cooling system of the opto-fluidic instrument or system, and the cooling system may include a network of coolant-carrying tubes that are configured to transport coolants to various modules of the opto-fluidic instrument or system for regulating the temperatures thereof. In such cases, the fluidics module may include coolant reservoirs for storing the coolants and pumps (e.g., “coolant pumps”) for generating a pressure differential, thereby forcing the coolants to flow from the reservoirs to the various modules of the opto-fluidic instrument or system via the coolant-carrying tubes. In some instances, the fluidics module may include returning coolant reservoirs that may be configured to receive and store returning coolants, e.g., heated coolants flowing back into the returning coolant reservoirs after absorbing heat discharged by the various modules of the opto-fluidic instrument or system. In such cases, the fluidics module may also include cooling fans that are configured to force air (e.g., cool and/or ambient air) into the returning coolant reservoirs to cool the heated coolants stored therein. In some instance, the fluidics module may also include cooling fans that are configured to force air directly into a component of the opto-fluidic instrument or system so as to cool said component. For example, the fluidics module may include cooling fans that are configured to direct cool or ambient air into the system controller to cool the same.

As discussed above, the opto-fluidic instrument or system may include an optics module which include the various optical components of the opto-fluidic instrument or system, such as but not limited to a camera, an illumination module (e.g., LEDs), an objective lens, and/or the like. The optics module may include a fluorescence imaging system that is configured to image the fluorescence emitted by the detectably labeled nucleotides are incorporated in extending sequencing primers in the sample after the detectable labels are excited by light from the illumination module of the optics module.

In some instances, the optics module may also include an optical frame onto which the camera, the illumination module, and/or the X-Y stage of the sample module may be mounted.

In various instances, the system controller may be configured to control the operations of the opto-fluidic instrument or system (e.g., and the operations of one or more modules thereof). In some instances, the system controller may take various forms, including a processor, a single computer (or computer system), or multiple computers in communication with each other. In various instances, the system controller may be communicatively coupled with data storage, set of input devices, display system, or a combination thereof. In some cases, some or all of these components may be considered to be part of or otherwise integrated with the system controller, may be separate components in communication with each other, or may be integrated together. In other examples, the system controller can be, or may be in communication with, a cloud computing platform.

In various instances, the opto-fluidic instrument or system may analyze the sample and may generate the output that includes indications of the presence of the target molecules in the sample. For instance, with respect to instances discussed above where the opto-fluidic instrument or system employs a sequencing technique for detecting molecules, the opto-fluidic instrument or system may cause the sample to undergo successive sequencing cycles, where during the same sequencing cycle the sample is imaged to detect signals associated with nucleotide binding and/or incorporation events at some locations in the sample, as well as to detect an absence of signals at other locations in the sample. In such cases, the output may include a series of optical signals (e.g., a code word) specific to each identifier sequence (e.g., a barcode sequence), which allow the identification of the target molecules.

### 4. Probes and Analytes

In some embodiments, methods disclosed herein include use of probe molecules (e.g., first probe and second probe) that are capable of being amplified. In some embodiments, the probe molecules (e.g., first probe and second probe) are capable of being circularized, e.g., to generate a circularized template for rolling circle amplification. In some cases, a probe (e.g., a first probe) comprises a 5′ end and a 3′ end and the 5′ end and 3′ end are ligated to generate a circularized template. In some embodiments, a circularizable probe (e.g., a second probe) is hybridized to a target nucleic acid molecule comprising a gap sequence. In some instances, the gap sequence comprises a variant sequence of interest. In some embodiments, the circularizable probe is circularized to generate a circularized probe comprising a gap filled sequence complementary to the gap sequence in the target nucleic acid molecule. In some embodiments, a second probe contacted with the biological sample is circularized to generate a circularized probe comprising a gap filled sequence complementary to the gap sequence in the target nucleic acid molecule. In some embodiments, the circularized template comprising at least portions of the complement of the gap sequence is amplified (e.g., through RCA) and the RCA product is detected in order to detect the variant sequence in the target nucleic acid molecule.

In some embodiments, the first probe is provided in one or more parts (e.g., one or more separate nucleic acid molecules). In some embodiments, the second probe is provided in one or more parts (e.g., one or more separate nucleic acid molecules). In some embodiments, the first probe is provided as at least two parts (e.g., at least two nucleic acid molecules). In some embodiments, the second probe is provided as at least two parts (e.g., at least two nucleic acid molecules). For example, a first part of the first probe comprises a first hybridization region and a second part of the first probe comprises a second hybridization region for binding to the target nucleic acid. In some instances, a first part of the second probe comprises a first hybridization region and a second part of the second probe comprises a second hybridization region for binding to the target nucleic acid, wherein the first and second hybridization regions are separated by a gap upon hybridization to the second target nucleic acid.

In some aspects, a first probe is capable of being amplified enzymatically or non-enzymatically. In some aspects, a first probe is selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes (e.g., as described in Section III).

In some aspects, the method comprises contacting the biological sample with a circularizable probe (e.g., a first probe or second probe) comprising a first hybridization region and a second hybridization region (e.g., 5′ and 3′ arms of a padlock probe) that hybridize to a first sequence and a second sequence, respectively, in a target nucleic acid (e.g., an RNA or cDNA) in the biological sample. In some embodiments, the first hybridization region and/or the second hybridization region in the circularizable probe comprises one or more RNA residues at and/or near its 3′ end. In some cases, a probe comprises a barcode sequence corresponding to the target nucleic acid or a sequence thereof. In some instances, a probe (e.g., a first probe) comprises from a 5′ end to a 3′ end: a first hybridization region—a barcode region comprising one or more barcode sequences
a second hybridization region. In some embodiments, the barcode sequence is associated with, corresponds to, and/or identifies a target nucleic acid or a sequence therein. In some aspects, upon hybridization to the target nucleic acid, the first hybridization region and the second hybridization region are positioned adjacent to each other for direct ligation. In some aspects, upon hybridization to the target nucleic acid, the 5′ end of the first probe is ligated to the 3′ end of the first probe using the first target nucleic acid as template.

In some embodiments, a circularizable probe (e.g., first probe) disclosed herein comprises one or more barcode sequences. In some embodiments, the first probe comprises one or more barcode sequences. In some embodiments, a circularizable probe comprises two or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some instances, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes can be independently and/or combinatorially detected and/or decoded. In some embodiments, the first probe does not contain a barcode sequence corresponding to the first target nucleic acid or a sequence thereof. In some embodiments, the first probe does not comprise any nucleic acid barcode sequence. In some embodiments, the first probe is detected by hybridizing, directly or indirectly, an oligonucleotide probe to a sequence of the first and/or second hybridization regions or a complement or portion thereof.

In some embodiments, the second probe does not contain a barcode sequence corresponding to the second target nucleic acid or a sequence thereof.

In some embodiments, the biological sample is contacted with a library of first probes to detect a panel of at least 50, at least 100, at least 200, at least 300, at least 400, at least 500, at least 1,000, at least 1,500, at least 2,000, at least 3,000, at least 4,000, at least 5,000 or more first target nucleic acids. In some embodiments, the biological sample is contacted with a library of first probes to detect a panel of at least 500 or more first target nucleic acids (e.g., mRNAs).

In some aspects, the method comprises contacting the biological sample with a circularizable probe (e.g., a second probe) comprising a first hybridization region and a second hybridization region (e.g., 5′ and 3′ arms of a padlock probe) that hybridize to a first sequence and a second sequence, respectively, in a target nucleic acid (e.g., an RNA or cDNA) in the biological sample, wherein the first sequence and the second sequence of the second target nucleic acid are separated by a gap sequence. In some cases, the gap sequence serves as a template for gap filling the probe. In some embodiments, the first and second hybridization regions are common among a plurality of circularizable probes that target a plurality of target nucleic acids that comprise different gap (e.g., variant) sequences. In some embodiments, each of the plurality of target nucleic acids comprises a common first target sequence (among the plurality of target nucleic acids) and a common second target sequence (among the plurality of target nucleic acids) that are complementary to the common first and second probe regions, respectively, among the plurality of circularizable probes. In some embodiments, a plurality of circularizable probes for gap filling comprise molecules of the same nucleic acid sequence. In some embodiments, the plurality of circularizable probes comprise molecules of different nucleic acid sequences. In some embodiments, any two or more different nucleic acid sequences of the circularizable probes comprise common first and second hybridization regions. In some cases, a plurality of probes (e.g., second probes) are capable of hybridizing to both a wildtype molecule and a mutant molecule of the target nucleic acid. For example, the target nucleic acid (e.g., second target nucleic acid) comprises from a 5′ end to a 3′ end: a first sequence for binding to a first hybridization region of a probe-a gap sequence-a second sequence for binding to a second hybridization region of the probe. In some instances, a second probe comprises from a 3′ end to a 5′ end: a first hybridization region-a gap sequence-a second hybridization region. In some instances, a second probe comprises a sequencing primer binding site. In some instances, a second probe comprises from a 3′ end to a 5′ end: a first hybridization region-a gap sequence-a second hybridization region. In some instances, a second probe comprises from a 3′ end to a 5′ end: a sequencing primer binding site-a first hybridization region-a gap sequence-a second hybridization region. In some instances, a second probe comprises from a 5′ end to a 3′ end: a sequencing primer binding site-a first hybridization region-a gap sequence-a second hybridization region.

In some cases, the first hybridization region and the second hybridization region in the first probe are equal in length. In some cases, the first hybridization region and the second hybridization region in the second probe are equal in length. In some cases, the first hybridization region is shorter or longer than the second hybridization region in the first probe and/or second probe. In some cases, the first hybridization region is shorter than the second hybridization region in the first probe. In some cases, the first hybridization region is longer than the second hybridization region in the first probe. In some cases, the first hybridization region is shorter than the second hybridization region in the second probe. In some cases, the first hybridization region is longer than the second hybridization region in the second probe. In some instances, the first hybridization region and/or the second hybridization region of the circularizable probe (e.g., first probe and/or second probe) is individually between about 5 and about 50 nucleotides in length. In some aspects, the first hybridization region and/or the second hybridization region of the circularizable probe (e.g., first probe and/or second probe) is individually between about 15 and about 25 nucleotides in length. In some aspects, the first hybridization region and/or the second hybridization region of the circularizable probe is individually between about 6 and about 18 nucleotides in length.

In some embodiments, a gap sequence of a target nucleic acid (e.g., second nucleic acid) comprises a nucleotide variation, a nucleotide polymorphism, a mutation, a substitution, an insertion, a deletion, a translocation, a duplication, an inversion, a rearrangement and/or a repetitive sequence, for identifying a variant sequence among a plurality of different sequences in situ in a biological sample. In some embodiments, the gap sequence comprises variant sequence of a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, a gap sequence of a target nucleic acid (e.g., second nucleic acid) comprises one or more exon-exon boundaries. In some embodiments, a gap sequence of a target nucleic acid (e.g., second nucleic acid) comprises a sequence of an exon-exon boundary.

In some embodiments, circularizable probes (e.g., second probe) that target common regions adjacent to hotspots for mutation are used. In some embodiments, the common regions flank a gap sequence in the target nucleic acid (e.g., second target nucleic acid). In some embodiments, the gap sequence comprises one or more hotspots for mutation. In some embodiments, the gap sequence comprises a variant sequence among a plurality of different variant sequences. In some embodiments, gaps in the circularizable probes upon hybridization to their nucleic acid targets are filled by polymerization (e.g., as described in Section II.A). In some embodiments, the gaps are filled by splint ligation, using a library of splint oligonucleotides that are diverse in sequences and comprise a plurality of possible variant sequences (e.g., possible mutations for the hotspots). In some embodiments, the library of splint oligonucleotides are incubated with the sample for hybridization to target nucleic acid molecules, allowing the best matching splint oligonucleotide to outcompete other splint oligonucleotides in the library (e.g., as described in Section II.B). In some embodiments, after washing the sample, the best matching splint oligonucleotides are ligated into the circularizable probes and the circularized probes are amplified.

In some embodiments, the first and second sequences in the target nucleic acid (e.g., second target nucleic acid) bound by the first hybridization region and second hybridization region of the circularizable probe are separated by a gap sequence in the target nucleic acid. In some embodiments, the gap sequence is about or at least 2, about or at least 4, about or at least 6, about or at least 8, about or at least 10, about or at least 12, about or at least 14, about or at least 16, about or at least 18, about or at least 20, or more nucleotides in length. In some instances, the gap sequence is between about 2 and about 40 nucleotides in length. In some cases, the target nucleic acid comprises a variant sequence at the 3′ or 5′ end of the gap sequence. In some cases, the target nucleic acid comprises a variant sequence at or near the central nucleotide(s) of the gap sequence. In some instances, the target nucleic acid comprises a variant sequence at least 1, 2, 3, 4, 5, 6, 7, 8, 9, or more phosphodiester bonds from the 3′ or 5′ end of the gap sequence.

In some embodiments, the gap sequence comprises a variant sequence among a plurality of different variant sequences. In some embodiments, the plurality of circularizable probes that target a plurality of target nucleic acids that comprise different variant sequences do not hybridize to the gap sequences (which comprise the different variant sequences), and instead hybridize to common first and second sequences that flank the gap sequence.

In some embodiments, the gap sequence comprises a barcode sequence. In some embodiments, the second target nucleic acid comprises a barcode sequence. For example, the barcode sequence comprises one barcode sequence among a plurality of different barcode sequences of an exogenous library of nucleic acid constructs introduced to the biological sample. In some aspects, the barcode sequence is used for lineage tracing. In some aspects, the biological sample is contacted with a library of constructs, wherein the constructs comprise barcoded nucleic acid molecules, before the plurality of circularizable probes (comprising the first probes and second probes) are bound to the target nucleic acids. In some embodiments, the second target nucleic acid comprises a unique barcode sequence or a unique collection of barcode sequences specific to the second target nucleic acid. In some instances, the second target nucleic acid is or is derived from a construct (e.g., a vector) comprising a barcode sequence and the gap sequence of the second target nucleic acid comprises the barcode sequence of the construct or a complement thereof.

In some embodiments, the gap sequence comprises a sequence of an immune molecule. In TCR and BCR RNA transcripts, the V(D)J sequences are 5′ to the constant region exon(s) and the 3′ poly(A) tail of the transcript. The number of V(D)J transcripts of a particular V(D)J join sequence in a sample comprising T cells or B cells of various antigen specificities can be low, and sequence information in particular V(D)J joins can be lost and become unavailable for subsequent in situ detection. In some embodiments, the present disclosure provides methods for high-throughput profiling of V(D)J transcripts in a large number of clonal T cell populations comprising TCRs with varying antigenic specificities. The methods and compositions disclosed herein may be used in research, diagnostics, and drug target discovery. Analyzing the spatial distribution of V(D)J transcripts in situ in various tissues could be used for development of therapeutic and/or prophylactic agents, e.g., TCR therapeutic treatment modalities and/or anti-disease vaccination.

Provided herein is a method for analysis of immune molecule sequences by contacting the biological sample with a circularizable probe (e.g., second probe), extending the second probe using a sequence of an immune molecule as template to generate an extended probe comprising a gap filled sequence, ligating the extended probe, performing rolling circle amplification, and sequencing the amplification product to determine the gap filled sequence or a complement thereof to detect the sequence of the immune molecule. In some aspects, the provided methods for immune molecule analysis allows for sensitive detection even if the number of particular transcripts are low in the biological sample. In some embodiments, the second target nucleic acid comprises a sequence of an immune molecule. In some aspects, the second target nucleic acids in a biological sample are highly variable. For example, the sequence of the immune molecule is an antigen receptor transcript. In some cases, the antigen receptor transcript is a T cell receptor (TCR) transcript, optionally wherein the TCR transcript comprises a TCRα VJ join, a TCRβ VDJ join, a TCRγ VJ join, or a TCRδ VDJ join. In some cases, the antigen receptor transcript is an immunoglobulin (Ig) transcript, optionally wherein the Ig transcript comprises an Igκ VJ join, an Igλ VJ join, or an IgH VDJ join. In some embodiments, the methods are used for identifying multiple different antigen receptor transcripts present at a plurality of locations in the biological sample.

In some embodiments, the second probes comprise gap fill circularizable probes that target conserved regions in the V and J sequences and the probes are gap filled to fill in the D sequence in order to identify the D region sequences. In some embodiments, gaps can be filled by polymerization, e.g., primer extension by a DNA polymerase using the 3′ end of a circularizable probe as a primer and a cDNA comprising a VDJ join as a template. In some embodiments, gaps can be filled by splint ligation, using a diversity library of gap fill oligonucleotides that comprises numerous possible D sequence variants. In some embodiments, a library of oligonucleotides are incubated with the sample for hybridization to cDNA molecules comprising VDJ joins, allowing the best matching oligonucleotide to outcompete other oligonucleotides in the library and hybridize to the corresponding VDJ sequence. After washing the sample, the best matching oligonucleotides can be ligated into the gap fill circularizable probes and the circularized probes can be amplified. In some embodiments, amplicons (e.g., RCA products) comprising V(D)J sequences or complements thereof are detected in situ using sequencing.

In some aspects, a nucleic molecule (e.g., second target nucleic acid) comprising a V(D)J join sequence disclosed herein, e.g., a cDNA, is a product of a TCR transcript. There are two subsets of T cells based on the exact pair of receptor chains expressed. These are either the alpha (α) and beta (β) chain pair, or the gamma (γ) and delta (δ) chain pair, identifying the αβ or γδ T cells, respectively. The expression of the β and δ chain is limited to one chain in each of their respective subsets and this is referred to as allelic exclusion. These two chains are also characterized by the use of an additional DNA segment—the diversity (D) region—during the rearrangement process. The D region is flanked by N nucleotides which constitutes the NDN region of the CDR3 in these two chains. In some aspects, the CDR3 of each of the two receptor chains defines the T cell clonotype of cells expressing TCRs comprising the CDR3. For αβ T cells the CDR3 is in most contact with the peptide bound to the MHC; as such, CDR3 sequences are generally a focus for analyzing immunological sequences. In some embodiments, the TCR transcript disclosed herein comprises a TCRα VJ join. In some embodiments, the TCR transcript disclosed herein comprises a TCRβ VDJ join. In some embodiments, the TCR transcript disclosed herein comprises a TCRγ VJ join. In some embodiments, the TCR transcript disclosed herein comprises a TCRδ VDJ join.

In some aspects, a nucleic molecule (e.g., second target nucleic acid) comprising a V(D)J join sequence disclosed herein, e.g., a cDNA, is a product of a BCR or immunoglobulin transcript. B cells are highly diverse, each expressing a practically unique BCR or immunoglobulin. There are approximately 10¹⁰-10¹¹ B cells in a human adult. Each B cell in an organism (e.g., human) expresses a different BCR that allows it to recognize a particular set of molecular patterns. Individual B cells gain this specificity during their development in the bone marrow, where they undergo a somatic rearrangement process that combines multiple germline-encoded gene segments to procures the BCR. Human BCR and antibody molecules are composed of heavy and light chains (each of which contains both constant (C) and variable (V) regions), which are encoded by genes on three loci: the immunoglobulin heavy locus IgH, containing the gene segments for the immunoglobulin heavy chain; the immunoglobulin kappa (κ) locus (Igκ), containing the gene segments for the κ light chain; and the immunoglobulin lambda (κ) locus (Ig λ), containing the gene segments for the λ light chain. Each heavy chain and light chain gene contains multiple copies of three different types of gene segments for the variable regions of the antibody proteins. For example, the human immunoglobulin heavy chain region contains Constant (e.g., Cμ and Cδ) gene segments and 44 Variable (V) gene segments plus 27 Diversity (D) gene segments and 6 Joining (J) gene segments. The light chains also possess Constant (e.g., Cμ and Cδ) gene segments and numerous V and J gene segments, but do not have D gene segments. DNA rearrangement causes one copy of each type of gene segment to go in any given lymphocyte, generating an enormous antibody repertoire, although some are removed due to self-reactivity.

Because of the rearrangement undergone of the V(D)J segment in T cells and B cells, only parts of the V(D)J segments (the V, D, and J segments) can be traced back to segments encoded in highly repetitive regions of the germline that are not typically sequenced directly from the germ line DNA. Furthermore, the V, D, and J segments can be significantly modified during the V(D)J rearrangement process and through, in the case of B cells, somatic hypermutation (SHM). As such, there are typically no pre-existing full-length templates to align to sequence reads of the V(D)J segments of T cell receptors and B cell immunoglobulins. In some embodiments, clonal grouping or clonotyping can involve clustering the set of V(D)J sequences into clones, which are defined as a group of cells that are descended from a common ancestor. Unlike the case of T cells, members of a B cell clone may differ in their V(D)J sequences due to SHM.

In addition to V(D)J recombination and SHM, gene rearrangements editing the immunoglobulin (Ig) genes include class switch recombination (CSR). Like V(D)J recombination, CSR requires the formation of DNA double strand breaks (DSBs) as the key initiating step. Under physiological conditions, DSBs are introduced at the Ig genes by the activity of B lymphocyte cell specific enzymes such as recombinase activating gene 1/2 (RAG1/2, for V(D)J recombination) and activation-induced cytidine deaminase (AID, for CSR). During CSR, AID generates DSBs in the Ig locus by targeting repetitive sequences in the switch (S) regions that precede each Ig heavy (IgH) coding sequence. Paired DSBs in the switch regions are then joined by the classical and alternative non-homologous end joining (NHEJ) pathways to generate a switch of the IgH. This long range joining is thought to be part of a general mechanism of DNA repair where two DSBs are joined in cis over long chromosome distances.

In the immature B cell's antibody immunoglobulin (Ig) heavy chain (IgH) locus, the order of arrangement of the nucleic acid sequence encoding the heavy chain segments (order of the heavy chain exons) are as follows: for human, they are (for IgM), 6 (for IgD), 73 (for IgG3), γ1 (for IgG1), α1 (for IgAQ1), γ2 (for IgG2), γ4 (for IgG4), F (for IgE), and α2 (for IgA2); and for mouse, they are μ (for IgM), δ (for IgD), γ3 (for IgG3), γ1 (for IgG1), γ2b (for IgG2b), γ2a (for IgG2a), ε (for IgE), and α (for IgA). Class switching occurs after the activation of a mature B cell via its membrane-bound antibody molecule (BCRs) to generate the different classes of antibodies. Ligand or antigen binding to the cell surface BCR triggers an intracellular cell signaling process that brings about CSR and produces the various classes of antibodies. The various classes of antibodies all have the same variable domains as the original antibody generated in the immature B cell during the process of V(D)J recombination, but possessing distinct constant domains in their heavy chains.

In some embodiments, disclosed herein is a method involving detecting a sequence of a V(D)J transcript in a biological sample. In some embodiments, V(D)J sequences include those in V(D)J transcripts comprising V(D)J joins. In some embodiments, V(D)J sequences include those in cDNA generated from V(D)J transcripts comprising V(D)J joins. In some embodiments, V(D)J sequences are incorporated into circularized probes generated during gap fill polymerization or gap fill oligonucleotide ligation. In some embodiments, V(D)J sequences are present in amplification products of V(D)J transcripts, amplification products of cDNA of V(D)J transcripts, or amplification products of the probes that hybridize to V(D)J transcripts or cDNA of V(D)J transcripts.

In some aspects, methods provided herein further comprise generating rolling circle amplification (RCA) products of the circularized probes extended with a sequence of an immune molecule and corresponding products thereof (e.g., RCA products) are detected for analyzing the spatial organization of V(D)J sequences in samples (e.g., tissues such as tumors comprising infiltrating immune cells). Such insights can be crucial to understanding disease development and establishing new treatment strategies.

In some embodiments, a probe disclosed herein (e.g., a second probe) includes a sequence complementary to a region of a target nucleic acid sequence encoding a constant region of an antibody or a fragment thereof. In some embodiments, a probe disclosed herein (e.g., a second probe) comprises a sequence complementary to a region of a target nucleic acid sequence encoding a constant region of an immune cell receptor. In some embodiments, a probe disclosed herein (e.g., a second probe) includes a sequence complementary to a region of a target nucleic acid sequence encoding a constant region of a B cell receptor. In some embodiments, a probe disclosed herein (e.g., a second probe) includes a sequence complementary to a region of a target nucleic acid sequence encoding a constant region of a T cell receptor.

Provided herein is a method for analysis of one or more perturbation agents introduced to a cell by contacting the biological sample with a circularizable probe (e.g., second probe), extending the second probe using a sequence of the perturbation agent or a corresponding molecule as template to generate an extended probe comprising a gap filled sequence, ligating the extended probe, performing amplification, and sequencing the amplification product to determine the gap filled sequence or a complement thereof to detect the sequence of the perturbation agent. In some aspects, the biological sample is contacted with a library of perturbation agents. In some aspects, the assays described herein are used for detecting CRISPR guides, e.g., guide RNAs (gRNAs). In some embodiments, the assays described herein are used for detecting perturbations introduced by CRISPR libraries and/or cellular RNA transcripts. In some cases, a sequence of a CRISPR guide RNA is incorporated by gap fill into a circularizable probe (e.g., second probe) by incorporating a sequence complementarity to the CRISPR guide RNA.

In some embodiments, the second target nucleic acid comprises a sequence of a perturbation agent or a corresponding molecule (e.g., a precursor or derivative thereof) as template for the gap fill of the circularizable probe. For example, the second target nucleic acid comprises a sequence of or associated with a perturbation agent introduced to the biological sample before circularizable probes are introduced. In some embodiments, a CRISPR molecule (e.g., a CRISPR RNA), a nucleic acid molecule edited using the CRISPR molecule, and/or a precursor or derivative thereof is detected. In some aspects, the second target nucleic acid comprises a sequence of a CRISPR molecule (e.g., a CRISPR RNA), a nucleic acid molecule edited using the CRISPR molecule, and/or a precursor or derivative thereof. In some instances, the second target nucleic acid is an RNA molecule derived from an exogenously introduced nucleic acid molecule. In some embodiments, the exogenously introduced nucleic acid molecule is an RNA derived from a plasmid, an integrated DNA sequence (e.g. using viral transduction in a cell), a gRNA from a CRISPR genetic element, etc. In some embodiments, the perturbation agent comprises a spacer sequence that is an element (e.g., about 20 nucleotides) that can be found as a component of gRNA. In some aspects, the spacer sequence found on the gRNA corresponds to a protospacer sequence that is found in the target region. The target region (the protospacer sequence) is the region of interest, e.g., a region of the cellular DNA designed to be targeted by the guide RNA. In some examples, the protospacer is found in the cellular DNA that is complementary to the protospacer that is found in the guide RNA. CRISPR enzymes can target a nucleic acid molecule using a guide RNA containing a spacer sequence that hybridizes to a target sequence of the nucleic acid molecule site. A CRISPR enzyme can be a Cas fusion protein. The system for introducing perturbations may further comprise a CRISPR enzyme. In some aspects, a CRISPR RNA comprises a spacer sequence to anneal to a target nucleic acid molecule and a scaffold sequence to bind to the Cas fusion protein. In some embodiments, a CRISPR RNA comprises a barcode sequence or a sequence for binding to a barcoded nucleic acid molecule.

In some embodiments, a circularizable probe (e.g., second probe) binds to or hybridizes to a protospacer sequence or hybridization regions flanking a protospacer sequence, or a complement thereof. In some embodiments, a circularizable probe (e.g., second probe) binds to a conserved region of the guide RNA (e.g., a common sequence shared by a plurality of different guide RNAs). In some aspects, CRISPR libraries are generated in cells of a biological sample. In some aspects, a CRISPR library may comprise hundreds, thousands, or tens of thousands of different spacer sequences. In some embodiments, a circularizable probe (e.g., second probe) hybridizes to the complement or reverse complement of a guide RNA spacer sequence. In some examples, a nucleic acid molecule to be analyzed is introduced and/or delivered into a cell or a cell constituent (e.g., a nucleus of a cell) using any of a variety of techniques.

In some embodiments, the second target nucleic acid is a transcript comprising a unique barcode specific to the perturbation agent. In some embodiments, the second target nucleic acid is a transcript comprising a unique barcode specific to a guide RNA. In some embodiments, the second target nucleic acid is a transcript comprising a guide RNA sequence. In some instances, a guide RNA and guide RNA barcode is expressed from the same vector and the barcode or a complement thereof is used as template to generate an extended probe comprising a gap filled sequence. For example, perturbation agents are described in U.S. Patent Application Publication No 2021/0171938.

In some embodiments, upon hybridization to the target nucleic acid, the circularizable probe is circularized to generate a circularized probe comprising a gap filled region complementary to the gap sequence. In some cases, the gap filled region is generated using gap filling by polymerization, or gap fill splint ligation, or a combination thereof. In some embodiments, a rolling circle amplification product (RCP) of the circularized probe is generated in the biological sample, and the RCP comprises multiple copies of the gap sequence or complement thereof.

In some embodiments, the circularizable probe (e.g., second probe) comprises a 5′ region and a 3′ region that hybridize to sequences adjacent to a gap sequence in the target nucleic acid. In some embodiments, upon hybridization of a circularizable probe to the target nucleic acid molecule, the 3′ terminal nucleotide and the 5′ terminal nucleotide of the circularizable probe are not juxtaposed directly next to each other; as such, a ligase alone cannot catalyze the formation of a phosphodiester bond directly between the 5′ phosphate group of the 5′ terminal nucleotide and the 3′ hydroxyl group of the 3′ terminal nucleotide. In some embodiments, upon hybridization of a circularizable probe to the target nucleic acid molecule, the 3′ terminal nucleotide and the 5′ terminal nucleotide of the circularizable probe are separated from each other by a gap of between about 1 and about 40 nucleotides in length. In some embodiments, the gap is about 2, about 5, about 10, about 15, about 20, about 25, about 30, about 35, about 40, about 45, about 50, or of any integer (or range of integers) of nucleotides in between the indicated values. In some embodiments, the gap is no more than about 40 nucleotides in length. In some embodiments, the gap is no more than about 30 nucleotides in length. In some embodiments, the gap is about 6, about 8, about 10, about 12, about 14, about 16, about 18, about 20, about 22, about 24, or of any integer (or range of integers) of nucleotides in between the indicated values. In some embodiments, the gap is no more than about 10 nucleotides in length. In some embodiments, the gap is about 5 nucleotides in length.

In some embodiments, a circularizable probe (e.g., second probe) disclosed herein does not comprise any nucleic acid barcode sequence. In some embodiments, circularizable probes for hybridizing to multiple different target nucleic acids comprise a common sequence that is not complementary to the target nucleic acids. For instance, the backbone sequences of a plurality of circularizable probes for detecting different variant sequences of a target nucleic acid is a common backbone sequence. In some embodiments, the backbone sequences of the plurality of gap fill padlock probes do not contain any nucleic acid barcode sequence that uniquely corresponds to a particular target nucleic acid or a particular sequence variant thereof.

In some embodiments, hybridization of substantially complementary or complementary nucleic acid sequences within two different molecules is analyzed. For example, hybridization of an endogenous analyte with a probe is analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes can be hybridized to an endogenous analyte and/or a labeling agent and each probe may comprise one or more barcode sequences. For example, barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe design can vary.

### Gap Fill Polymerization

In some embodiments, a gap in a circularizable probe (e.g., second probe) hybridized to the target nucleic acid molecule may be filled by extending a 3′ end of the circularizable probe to generate an extended probe comprising a gap filled sequence. In some embodiments, a polymerase is used to extend the 3′ end using the target nucleic acid molecule as a template, thereby filling the gap using the nucleotide sequence in the target nucleic acid molecule. In some embodiments, gap filling by the polymerase incorporates nucleotides residues into the circularizable probe, and the incorporated nucleotide sequence is complementary to the gap sequence or a portion thereof in the target nucleic acid molecule. In some embodiments, a polymerase is used to extend the 3′ end using the gap sequence in the target nucleic acid molecule as a template, thereby filling the gap using the nucleotide sequence in the target nucleic acid molecule.

In some instances, the gap filling is performed using a polymerase (e.g., DNA polymerase) in the presence of appropriate dNTPs and other cofactors, under isothermal conditions or non-isothermal conditions. Exemplary DNA polymerases include but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some instances, the gap filling is performed using a DNA polymerase capable of incorporating at least about 25, at least about 50, at least about 100, at least about 125, at least about 150, at least about 175, at least about 200, at least about 225, at least about 250, at least about 300, at least about 400, at least about 500, at least about 600, or at least about 1,000 nucleotides in a single binding event before dissociating from the target nucleic acid molecule.

Incorporation of the correct nucleotides to a growing strand of DNA, as determined by the template, is known as sequence fidelity. In some embodiments, a high fidelity DNA polymerase is used for gap filling and examples include but are not limited to: Taq DNA polymerase, Phusion® High-Fidelity DNA Polymerase, KAPA Taq, KAPA Taq HotStart DNA Polymerase, KAPA HiFi, and/or Q5® High-Fidelity DNA Polymerase.

In some instances, the gap filling is performed using a polymerase having no or limited strand displacement activity, such that an extended 3′ region of the circularizable probe does not displace the 5′ region hybridized to the nucleic acid molecule. For example, T4 and T7 DNA Polymerases lack strand displacement activity and can be used for this purpose. In some embodiments, especially where the target nucleic acid is RNA, the polymerase can be a reverse transcriptase. Reverse transcriptases having reduced strand displacement activity can be used, see, e.g., Martin-Alonso et al., ACS Infect. Dis. 2020, 6, 5, 1140-1153, which is incorporated herein by reference in its entirety.

In some embodiments, the 3′ region of the circularizable probe extended by the polymerase is juxtaposed to the 5′ region of the circularizable probe (e.g., second probe). In some embodiments, the 3′ region of the circularizable probe extended by the polymerase is juxtaposed to the 5′ region of the circularizable probe, forming a nick. In some embodiments, the ligation involves template dependent ligation, e.g., using the gap sequence in the target nucleic acid as template. In some embodiments, the ligation involves template independent ligation. The nick can be ligated using chemical ligation. In some embodiments, the chemical ligation involves click chemistry.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. In some aspects, the ligase used herein is a DNA ligase. In some aspects, the ligase used herein is an ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, AMPLIGASE™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase is a ssDNA ligase. In some embodiments, the ssDNA ligase is a bacteriophage TS2126 RNA ligase or an archaebacterium RNA ligase or a variant or derivative thereof. In some embodiments, the ligase is Methanobacterium thermoautotrophicum RNA ligase 1, CIRCLIGASE™ I, CIRCLIGASE™ II, T4 RNA ligase 1, or T4 RNA ligase 2, or a variant or derivative thereof.

### Splint Oligonucleotide Ligation

In some embodiments, a gap in a circularizable probe (e.g., second probe) hybridized to the target nucleic acid molecule is filled by a splint oligonucleotide. In some embodiments, the splint oligonucleotide is ligated to a circularizable probe to generate an extended probe comprising a gap filled sequence.

In some embodiments, the splint oligonucleotide comprises a sequence complementary to a nucleotide variation, a nucleotide polymorphism, a mutation, a substitution, an insertion, a deletion, a translocation, a duplication, an inversion, and/or a repetitive sequence, for identifying a variant sequence among a plurality of different sequences in situ in a biological sample. In some embodiments, the splint oligonucleotide can comprise a sequence complementary to a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the splint oligonucleotide can comprise a sequence complementary to a sequence comprising multiple nucleotides, and each nucleotide can be independently at the position of an SNV, an SNP, a point mutation, a single nucleotide substitution, a single nucleotide insertion, or a single nucleotide deletion. In some embodiments, the splint oligonucleotide comprises a sequence complementary to a sequence of or associated with an immune molecule. In some embodiments, the splint oligonucleotide comprises a sequence complementary to a sequence of or associated with a perturbation agent.

In some embodiments, provided herein is a library of splint oligonucleotides comprising i) a splint oligonucleotide comprising a sequence complementary to a nucleotide variation, a nucleotide polymorphism, a mutation, a substitution, an insertion, a deletion, a translocation, a duplication, an inversion, and/or a repetitive sequence, and ii) another splint oligonucleotide which does not comprise a sequence complementary to the nucleotide variation, nucleotide polymorphism, mutation, substitution, insertion, deletion, translocation, duplication, inversion, and/or repetitive sequence. In some embodiments, the library of splint oligonucleotides can comprise i) a splint oligonucleotide comprising a sequence complementary to a variant sequence or deletion or insertion, and ii) another splint oligonucleotide which does not comprise a sequence complementary to the variant sequence or deletion or insertion. For example, wildtype and variant splint oligonucleotides in the library, when contacted with the biological sample, can compete with one another for hybridization to a gap sequence comprising a variant sequence, and the complementary variant splint oligonucleotide can outcompete the wildtype splint oligonucleotide which is not complementary to the variant sequence (e.g., one or more nucleotides) in the gap sequence. The competition among splint oligonucleotides can allow the use of short (e.g., 2 nucleotides) splint oligonucleotides, while achieving specificity of splint oligonucleotide hybridization and/or ligation, for instance, when splint oligonucleotide hybridization and ligation are performed in the same reaction mix and/or the same reaction condition. In some embodiments, using a low hybridization temperature, less denaturation, and/or more co-factors such as Mg²⁺ or other factors that promote hybridization can enable the use of shorter splint oligonucleotides.

In some embodiments, upon hybridization to the target nucleic acid molecule, the 5′ terminal nucleotide of the splint oligonucleotide is adjacent to the 3′ terminal nucleotide of the circularizable probe, and the 3′ terminal nucleotide of the splint oligonucleotide is adjacent to the 5′ terminal nucleotide of the circularizable probe. In some embodiments, the 5′ terminal nucleotide of the splint oligonucleotide and the 3′ terminal nucleotide of the circularizable probe are separated by a nick or a gap of one or more nucleotides, e.g., 1, 2, 3, 4, 5, 6, 7, 8, 9, 10, or more nucleotides. In some embodiments, the 3′ terminal nucleotide of the splint oligonucleotide and the 5′ terminal nucleotide of the circularizable probe are separated by a nick or a gap of one or more nucleotides, e.g., 1, 2, 3, 4, 5, 6, 7, 8, 9, 10, or more nucleotides. The nick can be ligated using any suitable ligase disclosed herein, and the gap can be filled using any suitable ligase polymerase followed by ligation.

In some embodiments, upon hybridization to the target nucleic acid, the circularizable probe is circularized to generate an extended probe comprising a gap filled region complementary to the gap sequence.

In some embodiments, the circularizable probe is hybridized to the target nucleic acid, followed by contacting the biological sample with a library of splint oligonucleotides that compete for hybridization to the target nucleic acid (e.g., hybridization to the gap sequence in the target nucleic acid). In some embodiments, the hybridization of a splint oligonucleotide to the target nucleic acid and the ligation of the splint oligonucleotide to the circularizable probe are performed sequentially, e.g., the splint oligonucleotide hybridization is performed in a reaction condition or reaction mix, and the splint oligonucleotide ligation is performed in a different reaction condition or different reaction mix. In some embodiments, the hybridization of a splint oligonucleotide to the target nucleic acid and the ligation of the splint oligonucleotide to the circularizable probe are performed in the same reaction condition or the same reaction mix. In some embodiments, any one or more of the splint oligonucleotides in the library are 2 nucleotides or more in length. In some embodiments, the library of splint oligonucleotides is used to detect different variant sequences of a single target nucleic acid within the biological sample. In some embodiments, the different variant sequences of the single target nucleic acid are at different positions in the gap sequence. In some embodiments, a circularizable probe comprises a first hybridization region and a second hybridization region that hybridize to the common first sequence and second sequence shared by target nucleic acids comprising different variant sequences.

In some embodiments, the circularizable probe and the library of splint oligonucleotides are contacted with the target nucleic acid at the same time, in the same reaction mix or separately. For example, the circularizable probe and the library of splint oligonucleotides are premixed before contacting the biological sample with the mixture. In another example, two separate compositions comprising the circularizable probe and the library of splint oligonucleotides, respectively, are contacted with the biological sample. In some embodiments, the hybridization of a splint oligonucleotide to the target nucleic acid and the ligation of the splint oligonucleotide to the circularizable probe are performed in the same reaction condition or the same reaction mix. In some embodiments, any one or more of the splint oligonucleotides in the library can be 2 nucleotides or more in length. In some embodiments, the library of splint oligonucleotides comprises at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different sequences. In some embodiments, the molar concentration of the library of splint oligonucleotides is about equal to or about 2, about 4, about 8, about 10, or more times the molar concentration of the circularizable probe.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the splint oligonucleotide comprises a sequence complementary to the gap sequence in the target nucleic acid molecule (e.g., second target nucleic acid). In some embodiments, the biological sample is contacted with a library of splint oligonucleotides. In some embodiments, the library comprises at least about 2, at least about 4, at least about 10, at least about 20, at least about 50, at least about 100, or more oligonucleotides of different sequences. In some embodiments, the sequence diversity of the splint oligonucleotides in the library is such that at least or about 80%, at least or about 85%, at least or about 90%, at least or about 95%, or about 100% of the possible variant sequences in the gap sequence of the target nucleic acid in a sample have corresponding splint oligonucleotides in the library, e.g., the splint oligonucleotides comprise sequences that are complementary to the variant sequences in the target nucleic acid.

In some embodiments, the gap filling is performed under conditions permissive for specific hybridization of a splint oligonucleotide to its complementary sequence in the gap sequence in the target nucleic acid molecule, and/or specific hybridization of a circularizable probe to the target nucleic acid molecule. In some embodiments, the circularizable probe comprises hybridization regions that hybridize to the target nucleic acid molecule at sequences outside the gap sequence (e.g., at constant region sequences flanking the gap sequence), whereas the variant sequences in the gap sequence are complementary to the splint oligonucleotides (e.g., wildtype or mutant) in the library. In some embodiments, the circularized probe is amplified by RCA (e.g., as described in Section II.C), and the RCA product comprises multiple copies of the gap sequence in the target nucleic acid, as shown in the bottom panel of FIG. 1. In some embodiments, a sequence in the gap sequence in the RCA product is determined in situ, e.g., by sequencing the gap sequence as described in Section III.

In some embodiments, the splint oligonucleotides is between about 6 and about 24 nucleotides in length. In some embodiments, any one or more of the splint oligonucleotides in the library is about 6, about 8, about 10, about 12, about 14, about 16, about 18, about 20, about 22, or about 24 nucleotides in length. Any two or more of the splint oligonucleotides in the library can have the same length or different lengths. In some embodiments, the splint oligonucleotides in the library can be of the same length.

In some embodiments, the variant sequence is at least 1, 2, 3, 4, 5, 6, 7, 8, 9, or more phosphodiester bonds from the 3′ or 5′ end of the gap sequence. In some embodiments, the sequence complementary to the variant sequence is at least 1, 2, 3, 4, 5, 6, 7, 8, 9, or more phosphodiester bonds from the 5′ or 3′ end of the splint oligonucleotide. In some embodiments, the variant sequence is at or near the central nucleotide(s) of the gap sequence. In some embodiments, the sequence complementary to the variant sequence is at or near the central nucleotide(s) of the splint oligonucleotide. In some embodiments, the sequence complementary to the variant sequence is at or near the central 1, 2, 3, 4, or 5 nucleotide(s) of the splint oligonucleotide. In some embodiments, the sequence complementary to the variant sequence is no more than 1, no more than 2, no more than 3, no more than 4, no more than 5, or no more than 6 nucleotides from the central nucleotide(s) of the splint oligonucleotide.

### Amplification

In some instances, the first amplification product generated using the first probe is a first RCA product comprising multiple copies of the barcode sequence (or complements thereof) of the first probe. In some instances, the second amplification product generated using the second probe is a second RCA product comprising multiple copies of the gap filled sequence (or complements thereof). In some embodiments, RCA is performed, and following formation of the circularized probes (e.g., first and second probes), in some instances, a primer oligonucleotide is added for amplification. In some instances, the primer oligonucleotide is added with the circularizable probe. In some instances, the primer oligonucleotide is added before or after the circularizable probe is contacted with the sample. In some instances, the primer oligonucleotide for amplification of the circularized template (e.g., circularized probe) comprises a sequence complementary to a target nucleic acid, as well as a sequence complementary to the circularizable probe that hybridizes to the target nucleic acid. In some embodiments, amplification of the circularized probe is primed by the target nucleic acid. In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some instances, a primer oligonucleotide for amplification of the circularized template comprises a single-stranded nucleic acid sequence having a 3′ end that can be used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. In some cases, the primer oligonucleotide is about 6 bases to about 100 bases, such as about 25 bases.

In some embodiments, amplification of the first and/or second circularized template is primed by the target nucleic acid (e.g., target RNA). The target nucleic acid can optionally be immobilized in the biological sample. In some embodiments, the target RNA is cleaved by an enzyme (e.g., RNase H). In some embodiments, the target nucleic acid is cleaved at a position downstream of the sequences bound to the circularized probe. In some aspects, the methods disclosed herein allow targeting of RNase H activity to a particular region in a target RNA that is adjacent to or overlapping with a target sequence for a probe. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe by about 1 to about 20 nucleotides or by about 8 to about 10 nucleotides. The cleaved target RNA itself can then be used to prime RCA of the circular probe generated from a circularized probe (e.g., target-primed RCA). In some cases, a plurality of nucleic acid oligonucleotides can be used to perform target-primed RCA for a plurality of different target nucleic acids.

In any of the embodiments herein, the biological sample is contacted with the RNase H (and optionally with the nucleic acid oligonucleotide) before or during formation of the circularized probe and/or circularized gap filled probe (e.g., as described in Section II). In some embodiments, the biological sample is contacted with the oligonucleotide and with the RNase H simultaneously or sequentially (in either order) before contacting the sample with the probe. In any of the embodiments herein, the biological sample is contacted with the RNase H (and optionally with the nucleic acid oligonucleotide) after formation of the circularized template from ligating the probe. In any of the embodiments herein, the RNase H comprises an RNase H1 and/or an RNAse H2. In some embodiments, RNase inactivating agents or inhibitors are added to the sample after cleaving the target RNA.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a circularizable probe), the circularized probe is rolling-circle amplified to generate a RCA product (e.g., RCP) containing multiple copies of the sequence of the circularized template.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the RCP-generating polymerase comprises a modified recombinant Phi29-type polymerase. In some embodiments, the RCP-generating polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Suitable polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some aspects, during the amplification step, modified nucleotides can be added to the reaction to incorporate the modified nucleotides in the amplification product (e.g., nanoball). Examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide reacts with an acrylic acid N-hydroxysuccinimide moiety. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on.

In some embodiments, the extension reaction mixture comprises a deoxynucleoside triphosphate (dNTP) or derivative, variant, or analogue thereof. In some embodiments, the primer extension reaction mixture can comprise a catalytic cofactor of the polymerase. In any of the preceding embodiments, the primer extension reaction mixture can comprise a catalytic di-cation, such as Mg²⁺ and/or Mn²⁺.

In some aspects, the RCA product is anchored to a polymer matrix. The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the RCA products are copolymerized and/or covalently attached to the surrounding matrix thereby preserving their spatial relationship and any information inherent thereto. In some embodiments, the RCA products are generated from DNA or RNA within a cell embedded in the matrix. In some embodiments, the RCA products are functionalized to form covalent attachment to the matrix preserving their spatial information within the cell thereby providing a subcellular localization distribution pattern. In some embodiments, the provided methods involve embedding RCA products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing or probe hybridization while an existing hydrogel-tissue chemistry method cannot. In some embodiments, to enable amplification product embedding in the tissue-hydrogel setting, amine-modified nucleotides are comprised in the RCA product, functionalized with an acrylamide moiety using acrylic acid N-hydroxysuccinimide esters, and copolymerized with acrylamide monomers to form a hydrogel.

In some embodiments, the generated amplification products of the first probe and the second probe are subject to analysis and/or sequence determination comprising detecting a sequence in all or a portion of the nucleic acid concatemer or in situ hybridization to the generated RCA products. In some embodiments, a first RCA product generated is subject to analysis and/or sequence determination comprising in situ hybridization to the RCP. In some embodiments, a second RCA product generated is subject to analysis and/or sequence determination comprising in situ sequencing of the RCP to determine the gap filled sequence or a complement thereof. In some embodiments, the detection involves contacting the biological sample with an oligonucleotide probe that directly or indirectly hybridize to a sequence in the first RCA product and detecting a first signal associated with the oligonucleotide probe, thereby detecting the first target nucleic acid, and sequencing the second RCA product to determine the gap filled sequence or a complement thereof, thereby detecting the second target nucleic acid. In some instances, the gap filled sequence or a complement thereof is detected by sequencing by ligation, sequencing by synthesis or sequencing by binding.

### 5. Compositions and Kits

In some aspects, provided herein are compositions comprising any of the nucleotides, primers, polymerases, and/or primary probes (e.g., circular probes or circularizable probes or probe sets) described herein.

In some instances, provided herein is a kit comprising any of the polymerase molecules described herein. In some instances, the kit further comprises any of the primers described herein. In some instances, provided herein is a kit further comprising any of the nucleotide molecules described herein.

In some instances, provided herein is a kit for sequencing comprising a plurality of polymerase molecules as described herein, and one or more additional reagents for performing the sequencing reaction. In some instances, the one or more additional reagents are selected from: a primer, nucleotide molecules, a flow cell, primers, and adapters for sequencing library preparation, or any combination thereof. In some embodiments, the polymerase is a polymerase permissive for a 3′ blocking group. In some embodiments, the one or more additional reagents include a primer, such as a sequencing primer.

In some instances, provided herein is a kit for performing in situ sequencing comprising a plurality of nucleotide molecules as described herein, and one or more additional reagents for performing the in situ sequencing reaction. In some instances, the one or more additional reagents include a polymerase, a primer, modified, a support for a tissue or cell sample (e.g., a slide), or any combination thereof. In some instances, the kit further comprises any of the circular probes and/or circularizable probes or probe sets disclosed herein. In some instances, the kit includes a polymerase for rolling circle amplification, and optionally dNTPs for the rolling circle amplification.

The various components of the kit may be present in separate containers or certain compatible components may be pre-combined into a single container. In some instances, the kits further contain instructions for using the components of the kit to practice the provided methods. In some instances, sets of nucleotide molecules having each nucleobase type (as described elsewhere) may be provided together in a single container, such as a tube. In some instances, the nucleotide molecules of each nucleobase type may be provided in separate containers. In some instances, a first combination of nucleotide molecules comprising (e.g., two of four nucleobase types) are provided together in a first container, and a second combination of nucleotide molecules (e.g., of the other two of four nucleobase types) may be provided in a second container.

In some aspects, provided herein is a kit for sequencing a template nucleic acid molecule, comprising: a plurality of nucleotide molecules as described herein; a primer designed to hybridize to the template nucleic acid molecule; and a polymerase. In some instances, the plurality of nucleotide molecules comprises four sets of nucleotide molecules, wherein each of the four sets of nucleotide molecules comprises a different nucleobase and a different dye. In some instances, molecules of three of the four different nucleotide molecules are coupled to different dyes, and molecules of one of the four different nucleobase types are not conjugated to a fluorophore.

In some embodiments, the kits contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits optionally contain other components, for example nucleic acid primers.

### 6. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

In the present description, the term “about” means ±20% of the indicated range, value, or structure, unless otherwise indicated.

Unless the context requires otherwise, throughout the present specification and claims, the word “comprise” and variations thereof, such as “comprises” and “comprising,” are to be construed in an open, inclusive sense, that is, as “including, but not limited to”. The term “Consisting of” in the context of consisting of one or more components or steps shall mean include only the one or more components or steps. The term “consisting essentially of” in the context of consisting essentially of one or more components or steps limits the components or steps to the specified materials or steps, or to those that do not materially affect the basic characteristics of a claimed invention.

As used herein, the terms “include” and “have” are used synonymously, which terms and variants thereof are intended to be construed as non-limiting.

All publications, comprising patent documents, scientific articles and databases, referred to in this application are incorporated by reference in their entirety for all purposes to the same extent as if each individual publication were individually incorporated by reference.

### EXAMPLES

The following examples are included for illustrative purposes only and are not intended to limit the scope of the present disclosure.

### Example 1: Modified Polymerases

This example describes engineered polymerases expressed and purified based on putative polymerase genes from three thermophilic species: Thermodesulfovibrio aggregans (UNIPROT ID: A0A0U9HY46, referred to as “9HY46”) a putative Stygiolobus azoricus UNIPROT ID: A0A650CR43, or “CR43”); and Thermofilum pendens (UniProt ID: A1RX72, “RX72”). Descriptions of the mutations engineered into these polymerases are provided in Table 1.

TABLE 1
Polymerases

| | SEQ ID | |
|---|---|---|
| Polymerase | NO | Mutations vs Wild Type |
| 9HY46 mut | 1 | D165A, E167A, L436Y, Y437A, P438V, |
| 1 | | A515L |
| 9HY46 mut | 2 | D165A, E167A, L436I, Y437A, A515E, |
| 2 | | Y412F, V417I, V612H, I706K, V710M |
| CR43 mut | 4 | S178A, Q180A, M433Y, Y434A, P435V, A509L |
| RX72 mut | 6 | D184A, E186A, M439Y, Y440A, P441V, A521L |

### Example 2: Recombinant Expression and Purification of Polymerase 1

This example describes recombinant expression and purification of the engineered polymerases shown in Table 1.

To express the polymerases, competent BL21 (DE3) gold E. coli cells were transformed with an expression plasmids encoding the polymerases. Transformed cells were cultured overnight at 18° C. and induced with 0.6 mM Isopropyl β-D-1-thiogalactopyranoside (IPTG). The induced cells were pelleted and lysed by a microfluidizer. The lysate was treated with 0.5% Polyethylenimine (PEI) to precipitate nucleic acids. Ammonium sulfate precipitation was performed to remove residual PEI. Generation of the protein product was confirmed by sodium dodecyl sulfate polyacrylamide gel electrophoresis (SDS-PAGE) gel analysis (not shown). Following removal of residual PEI by ammonium sulfate precipitation, the crude protein product was purified by nickel affinity chromatography using the AKTA PURE™ protein purification system (Cytivia Life Sciences, Marlborough, MA), heparin chromatography, and dialysis. Purification of the protein product was confirmed by SDS-PAGE gel analysis (not shown). RX72 and 9HY46 mutants were successfully purified, whereas only minimal protein (<0.5 mg) was obtained for the CR43 mutant.

### Example 3: Testing of Engineered Polymerases in a Single Nucleotide Incorporation Assay at Different Temperatures

This example describes testing of the engineered polymerases for incorporating a modified nucleotide molecule into a priming strand for a sequencing reaction at different temperatures.

The engineered polymerases were evaluated in single nucleotide incorporation assays, the design of which is shown in FIG. 2.

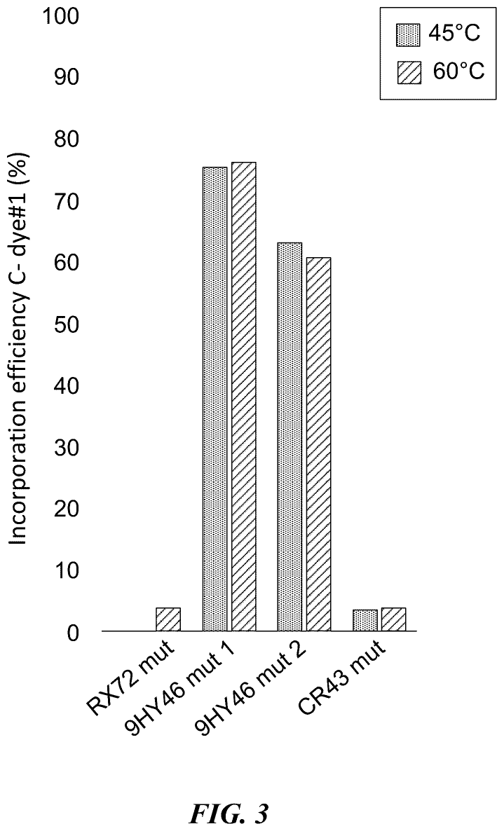

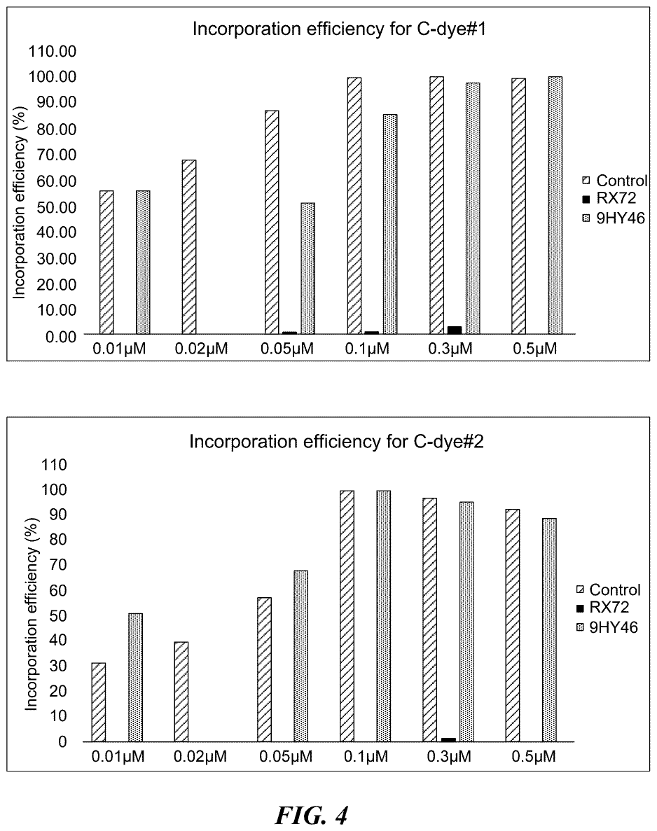

In a first incorporation assay, percent incorporation of fluorescently labelled cytosine nucleotides including a 3′ blocking group, following incubation with each engineered polymerase (0.5 uM) at 45° cand 60° C. was determined. As shown in FIG. 3 both mutant versions of 9HY46 showed significant incorporation of the nucleotide at both temperatures (at least 60% incorporation for each condition). In contrast, the CR43 mutant and RX72 mutant showed little to no incorporation. No incorporation was seen for the RX72 mutant at 45° C. In a second incorporation assay, various concentrations were tested for the RX72 mutant and 9HY46 mutant 1, using a fluorescently labelled, 3′ blocked cytosine nucleotide (“C-dye #1) and a fluorescently labelled, 3′ blocked guanine nucleotide (“G-dye #2). The incorporation reactions for each concentration were conducted at 50° C., and compared to a control 9° enzyme including mutations to residues homologous to those mutated in the RX72 mutant and 9HY46 mutant 1. As shown in FIG. 4, overall the incorporation efficiencies for 9HY46 mutant 1 were comparable to those of the control enzyme. Surprisingly, at the lowest tested concentration (0.01 uM), 9HY46 mutant 1 had 1.6 times higher incorporation efficiency for the modified guanine nucleotide, as compared to the control. Additionally, at 0.05 uM 9HY46 mutant 1 had 1.2 times higher incorporation efficiency for the modified guanine, as compared to the control. RX72 mutant showed little to no activity at each tested concentration for both modified nucleotides.

SEQUENCE LISTING

| NO. | DESCRIPTION | SEQUENCE |
|---|---|---|
| 1 | 9HY46 mut. 1 | MLFYPIDIDDSSEGILLFGITEDGRREKFIDSSYRPYFLVLPSD |
| | (D165A, | KKKAAAEIEKIVREKSLPVTEITEDKRIFYGQEREFIKIYTKR |
| | E167A, L436I, | HQDLQKVRDAIKILEAKRGGSGSIIDEFEYQISPYRMYLAEK |
| | Y437A, P438V, | QLSCLQWLEIETHRNQIKSIKKATANPPKLKILAFAMAVIEV |
| | A515L) | QRGTPSIVMISIFGEGLSRVITYQEAKYTIDATVVKNEKELIK |
| | | NFIENVRDYDPDIIVGYNTDLYDMPILRERAKQLKVDIGILS |
| | | RDNSGITLSKRARFTTARLIGRVHIDIFNFVFNILSPILQSE |
| | | QLTLKNVSQELLGDTKLEMEYEEILQAWDKGHELDKLARYCL |
| | | KDSELVYELSKMLLPDIMELTKITGQSLFDTSRMPYSQLVE |
| | | WYYIKKAKQQNRVIPNQPKFDEIKERQKITYEGGFVKEPEV |
| | | GLHKGIAVVDFASYAVSIIATYNISIDTLNCNCCKESGYKVP |
| | | DFPYWFCKNIRGFESQAIEELLMERLELKKQLKTLNPESHE |
| | | YLELDTKQKLLKTIINASYGYYAYPASRWYSKECAEAITAL |
| | | GRYWIKEVLTKAEQKGFHPIYADTDSAFLKIDSKEEVLQFI |
| | | KEINQSLPGVMRLDFEDFYVKGLFVPRQIGGVAKKRYALV |
| | | DEKGRLKIRGLEVVRRDLCRYARATQQEILRICLIEEDIPKA |
| | | FKYLDERIKALREGKYDLKDLVVYEQLSKPVSEYKLISPHVA |
| | | AAKRLLEKEIPVGEGSVIGYIIQKGSGSISERAYPIELAEPS |
| | | KIDIDYYIENQILPTAMRILKVFKKENKLF |
| 2 | 9HY46 mut. 2 | MLFYPIDIDDSSEGILLFGITEDGRREKFIDSSYRPYFLVLPSD |
| | (D165A, | KKKAAAEIEKIVREKSLPVTEITEDKRIFYGQEREFIKIYTKR |
| | E167A, L436I, | HQDLQKVRDAIKILEAKRGGSGSIIDEFEYQISPYRMYLAEK |
| | Y437A, A515E, | QLSCLQWLEIETHRNQIKSIKKATANPPKLKILAFAMAVIEV |
| | Y412F, V417I, | QRGTPSIVMISIFGEGLSRVITYQEAKYTIDATVVKNEKELIK |
| | V612H, 1706K, | NFIENVRDYDPDIIVGYNTDLYDMPILRERAKQLKVDIGILS |
| | V710M) | RDNSGITLSKRARFTTARLIGRVHIDIFNFVFNILSPILQSE |
| | | QLTLKNVSQELLGDTKLEMEYEEILQAWDKGHELDKLARYCL |
| | | KDSELVYELSKMLLPDIMELTKITGQSLFDTSRMPYSQLVE |
| | | WYYIKKAKQQNRVIPNQPKFDEIKERQKITFEGGFIKEPEVG |
| | | LHKGIAVVDFASIAPSIIATYNISIDTLNCNCCKESGYKVPDF |
| | | PYWFCKNIRGFESQAIEELLMERLELKKQLKTLNPESHEYL |
| | | ELDTKQKELKTIINASYGYYAYPASRWYSKECAEAITALGR |
| | | YWIKEVLTKAEQKGFHPIYADTDSAFLKIDSKEEVLQFIKEI |
| | | NQSLPGVMRLDFEDFYVKGLFHPRQIGGVAKKRYALVDEK |
| | | GRLKIRGLEVVRRDLCRYARATQQEILRICLIEEDIPKAFKY |
| | | LDERIKALREGKYDLKDLVVYEQLSKPVSEYKLKSPHMAAAK |
| | | RLLEKEIPVGEGSVIGYIIQKGSGSISERAYPIELAEPSKID |
| | | IDYYIENQILPTAMRILKVFKKENKLF |
| 3 | 9HY46 WT | MLFYPIDIDDSSEGILLFGITEDGRREKFIDSSYRPYFLVLP |
| | | SDKKKAAAEIEKIVREKSLPVTEITEDKRIFYGQEREFIKIY |
| | | TKRHQDLQKVRDAIKILEAKRGGSGSIIDEFEYQISPYRMYL |
| | | AEKQLSCLQWLEIETHRNQIKSIKKATANPPKLKILAFDMEV |
| | | IEVQRGTPSIVMISIFGEGLSRVITYQEAKYTIDATVVKNEK |
| | | ELIKNFIENVRDYDPDIIVGYNTDLYDMPILRERAKQLKVDI |
| | | GILSRDNSGITLSKRARFTTARLIGRVHIDIFNFVFNILSPI |
| | | LQSEQLTLKNVSQELLGDTKLEMEYEEILQAWDKGHELDKLA |
| | | RYCLKDSELVYELSKMLLPDIMELTKITGQSLFDTSRMPYSQ |
| | | LVEWYYIKKAKQQNRVIPNQPKFDEIKERQKITYEGGFVKEP |
| | | EVGLHKGIAVVDFASLYPSIIATYNISIDTLNCNCCKESGYK |
| | | VPDFPYWFCKNIRGFESQAIEELLMERLELKKQLKTLNPESH |
| | | EYLELDTKQKALKTIINASYGYYAYPASRWYSKECAEAITAL |
| | | GRYWIKEVLTKAEQKGFHPIYADTDSAFLKIDSKEEVLQFI |
| | | KEINQSLPGVMRLDFEDFYVKGLFVPRQIGGVAKKRYALVD |
| | | EKGRLKIRGLEVVRRDLCRYARATQQEILRICLIEEDIPKA |
| | | FKYLDERIKALREGKYDLKDLVVYEQLSKPVSEYKLISPHV |
| | | AAAKRLLEKEIPVGEGSVIGYIIQKGSGSISERAYPIELAE |
| | | PSKIDIDYYIENQILPTAMRILKVFKKENKLF |
| 4 | RX72 mut. | MVAGEVRELEFWILDASYDVVKGHPVIYLWGITESGERIV |
| | | VAEKNFRPYFYLVPENAISDALVERVRTALHTFKVLSVNA |
| | | IERKLFGRTLNVLKVTITDPRDVPKAREVAAKIPGIRDVL |
| | | EADIRFYMRYMVDTGISPSSWVLAKVRSAPVPNGWRVDEF |
| | | FELVEAPRSLDKKSTPRLKTYAFAIACYNRYGEPDPERDP |
| | | VLVISRTGEDGTVIFSQDEGSSEKKLLEEFVNDLVQYDPD |
| | | VILGYNSNRFDWPYLLQRARVNGVKLSIGRNLGEPSQSVY |
| | | GHFSVVGRANVDLYDYASELQEVKVKTLENVSEFLGVMKK |
| | | SERVLIDTNKVYEYWDSKDKRSLLLRYAGDDARSTYLLGQ |
| | | VVLPFGIQLSSLVGLPLDQVFAASVGNRVEWFLIRQAFVF |
| | | NELVPNSRERGEETYKGAIVLKPKPGVHKKIAVLDFSSYA |
| | | VNIMIKYNISPDTYVPPEEHVDPSEVWVAPEVGHRFRKHP |
| | | PGFYRKVLESLLEARRRLREKMKTLDPRSEEYRIYDERQK |
| | | LIKVITNATYGYSGWSMARWYKREVAEATTAWGRELIKAT |
| | | IKKAQSLGLSIIYGDTDSIFVQFDEEKIGRLVEYVEKELG |
| | | FEIKLDKVYEKVFFTESKKKYCGLLADGRVDLVGFEAVRG |
| | | DWAEISKEIQERVVEIVLKEEDPWKAVDFVRKTITELSEG |
| | | RIPLEKLVIWKTLSKDLDEYEVDAPHVVVARELKRLGYRV |
| | | GKGAKIGYIVVKGAGKVSEKAKPLISVKSPDEVDTDYYVK |
| | | RQIVPAALRILEYFGVKEEHFSTGRRQATLSDFF |
| 5 | (D184A, | MVAGEVRELEFWILDASYDVVKGHPVIYLWGITESGERIV |
| | E186A, | VAEKNFRPYFYLVPENAISDALVERVRTALHTFKVLSVNAI |
| | M439Y, | ERKLFGRTLNVLKVTITDPRDVPKAREVAAKIPGIRDVLEA |
| | Y440A, P441V, | DIRFYMRYMVDTGISPSSWVLAKVRSAPVPNGWRVDEFFE |
| | A521L) | LVEAPRSLDKKSTPRLKTYAFDIECYNRYGEPDPERDPVLVI |
| | RX72 WT | SRTGEDGTVIFSQDEGSSEKKLLEEFVNDLVQYDPDVILGY |
| | | NSNRFDWPYLLQRARVNGVKLSIGRNLGEPSQSVYGHFSV |
| | | VGRANVDLYDYASELQEVKVKTLENVSEFLGVMKKSERV |
| | | LIDTNKVYEYWDSKDKRSLLLRYAGDDARSTYLLGQVVLP |
| | | FGIQLSSLVGLPLDQVFAASVGNRVEWFLIRQAFVFNELVP |
| | | NSRERGEETYKGAIVLKPKPGVHKKIAVLDFSSMYPNIMIK |
| | | YNISPDTYVPPEEHVDPSEVWVAPEVGHRFRKHPPGFYRK |
| | | VLESLLEARRRLREKMKTLDPRSEEYRIYDERQKAIKVITN |
| | | ATYGYSGWSMARWYKREVAEATTAWGRELIKATIKKAQS |
| | | LGLSIIYGDTDSIFVQFDEEKIGRLVEYVEKELGFEIKLDKV |
| | | YEKVFFTESKKKYCGLLADGRVDLVGFEAVRGDWAEISKE |
| | | IQERVVEIVLKEEDPWKAVDFVRKTITELSEGRIPLEKLVIW |
| | | KTLSKDLDEYEVDAPHVVVARELKRLGYRVGKGAKIGYIV |
| | | VKGAGKVSEKAKPLISVKSPDEVDTDYYVKRQIVPAALRIL |
| | | EYFGVKEEHFSTGRRQATLSDFF |
| 6 | CR43 mut. | MIKDFFILDFSYDVIDNIPVIFIWAIDTEGQRIVLLEKSFRPYF |
| | (S178A, | YVVVDEKEVEYIIEEIKKLSKSGSPITNIIPIKGKKLFGQPVT |
| | Q180A, | VLRIETLIPAYVRIYRDEVLKVKGVKQVLEADIRFYMRYSI |
| | M433Y, | DKYIKPFHWIKAEVEEVENINFRVNKVYEIKNIKEQYVGSIP |
| | Y434A, | KLRKLSFAIAVYSKQGSPNPRKDPIVVIAVKTDDSAKQFVA |
| | P435V, | SDSDDKQIIKEFVAFFQEYDPDIVYGENTNSFDLQYLLERAH |
| | A509L) | SKGLKLDIGRRVNGEPSQGVYGHYSMTGRLNVDLAGFVNS |
| | | VSEVKIKTLENICDYLGIVKKEDRVNLEWYEIPKMWEDKD |
| | | KKNKVIQYVADNARSVYLLGESFLPFAIELTRITGLPLDQLS |
| | | MASVGYRVEWLLMRESNKYNELIPNKEERTGESYEGGLVI |
| | | SPIQGIHENVIVLDFSSYAVSIMIKYNVGPDTLVKGECNDC |
| | | WVSPIGHKFRKEPPGLYKFVLENLVKERREIKSKLKETKDE |
| | | YEKRLLDERQKLLKVMANAFYGYMGWINARWYSREGAE |
| | | AVTAWGRSIISDAAKIATEHGFKVIYGDTDSIFIKGNGDINK |
| | | LQEIISQKFGIEIKMDKVYKRVLFTESKKRYAGITEDGRIDIV |
| | | GFEAIRGDWCELAREVQRNVIEIILRTGKVDDAVRYVRKTI |
| | | LSLRRGEFKIEDLIIWKGLDKDVNEYAITAPHVVAAKKAIK |
| | | AGYLVTKGSKIGYVIVKGTNKLSERAEPYFLVKDNSRIDIS |
| | | YYVDNQIIPSAMHVLKVFGVKEDSLKSGGTDILSFFKK |
| 7 | CR43 WT | MIKDFFILDFSYDVIDNIPVIFIWAIDTEGQRIVLLEKSFRPYF |
| | | YVVVDEKEVEYIIEEIKKLSKSGSPITNIIPIKGKKLFGQPVT |
| | | VLRIETLIPAYVRIYRDEVLKVKGVKQVLEADIRFYMRYSI |
| | | DKYIKPFHWIKAEVEEVENINFRVNKVYEIKNIKEQYVGSIP |
| | | KLRKLSFSIQVYSKQGSPNPRKDPIVVIAVKTDDSAKQFVA |
| | | SDSDDKQIIKEFVAFFQEYDPDIVYGFNTNSFDLQYLLERAH |
| | | SKGLKLDIGRRVNGEPSQGVYGHYSMTGRLNVDLAGFVNS |
| | | VSEVKIKTLENICDYLGIVKKEDRVNLEWYEIPKMWEDKD |
| | | KKNKVIQYVADNARSVYLLGESFLPFAIELTRITGLPLDQLS |
| | | MASVGYRVEWLLMRESNKYNELIPNKEERTGESYEGGLVI |
| | | SPIQGIHENVIVLDFSSMYPSIMIKYNVGPDTLVKGECNDC |
| | | WVSPIGHKFRKEPPGLYKFVLENLVKERREIKSKLKETKDE |
| | | YEKRLLDERQKALKVMANAFYGYMGWINARWYSREGAE |
| | | AVTAWGRSIISDAAKIATEHGFKVIYGDTDSIFIKGNGDINK |
| | | LQEIISQKFGIEIKMDKVYKRVLFTESKKRYAGITEDGRIDIV |
| | | GFEAIRGDWCELAREVQRNVIEIILRTGKVDDAVRYVRKTI |
| | | LSLRRGEFKIEDLIIWKGLDKDVNEYAITAPHVVAAKKAIK |
| | | AGYLVTKGSKIGYVIVKGTNKLSERAEPYFLVKDNSRIDIS |
| | | YYVDNQIIPSAMHVLKVFGVKEDSLKSGGTDILSFFKK |

What is claimed is:

1. A modified polymerase comprising an amino acid sequence that is at least 80% identical to SEQ ID NO: 3 and comprising the following amino acid mutations:
an L436 mutation selected from L436Y, L436S, and L436T;
a Y437 mutation selected from Y437A and Y437G; and
at least one of: a D165A mutation and a E167A mutation.

2. The modified polymerase of claim 1, wherein the Y437 mutation is Y437A.

3. The modified polymerase of claim 1, wherein the L436 mutation is L436Y.

4. The modified polymerase of claim 1, comprising the D165A mutation and the E167A mutation.

5. The modified polymerase of claim 1, further comprising a P438 mutation selected from: P438V, P438L, and P431I.

6. The modified polymerase of claim 1, further comprising a A515L mutation.

7. The modified polymerase of claim 1, wherein the polymerase has the sequence of SEQ ID NO: 1.

8. The modified polymerase of claim 1, further comprising:
a Y412F mutation, a V417I mutation, a V612H mutation, an I706K mutation, or a V710M mutation.

9. The modified polymerase of claim 8, comprising:
a Y412F mutation, a V417I mutation, a V612H mutation, an I706K mutation, and a V710M mutation.

10. The modified polymerase of claim 1, wherein the polymerase has the sequence of SEQ ID NO: 2.

11. The modified polymerase of claim 1, comprising an amino acid sequence that is at least 85%, at least 90%, or at least 95% identical to SEQ ID NO: 3.

12. The modified polymerase of claim 1, wherein the polymerase is capable of incorporating modified nucleotides at a reaction temperature from about 30° C. to about 80° C.

13. The modified polymerase of claim 1, wherein the modified nucleotides each comprise a 3′ blocking group.

14. The modified polymerase of claim 1, wherein at least a subset of the modified nucleotides are linked to a dye.

15. The modified polymerase of claim 14, wherein the dye is a fluorescent dye.

16. The modified polymerase of claim 1, wherein the polymerase is capable of incorporating modified nucleotides in a sequencing-by-synthesis (SBS) reaction.

17. A method for sequencing a template nucleic acid molecule comprising:
(a) contacting a template nucleic acid molecule bound to a priming strand with: (i) the modified polymerase of claim 1 and (ii) a first plurality of modified nucleotide molecules comprising a 3′ blocking group, thereby incorporating a modified nucleotide molecule into the priming strand; and
(b) detecting a presence of the incorporated modified nucleotide molecule in the priming strand to identify a complementary nucleotide in the template nucleic acid molecule.

18. The method of claim 17, wherein the first plurality of modified nucleotide molecules includes:
(i) modified nucleotide molecules of a first nucleobase type, each attached to a first dye,
(ii) modified nucleotide molecules of a second nucleobase type, each attached to a second dye,
(iii) modified nucleotide molecules of a third nucleobase type, each attached to a third dye, and
(iv) modified nucleotide molecules of a fourth nucleobase type.

19. A method for sequencing a template nucleic acid molecule comprising:
a) contacting a template nucleic acid molecule bound to a priming strand with: (i) the modified polymerase of claim 1 and (ii) a first plurality of modified nucleotide molecules comprising a 3′ blocking group, thereby incorporating a modified nucleotide molecule into the priming strand,
b) detecting a presence of the incorporated modified nucleotide molecule in the priming strand to identify a complementary nucleotide in the template nucleic acid molecule,
c) unblocking the blocking group, and
d) contacting the priming strand bound to the template nucleic acid molecule with the modified polymerase of claim 1 and a second plurality of modified nucleotide molecules each comprising a 3′ blocking group, thereby incorporating a nucleotide molecule of the second plurality of nucleotide molecules into the priming strand.

20. A kit for sequencing a template nucleic acid molecule comprising: a plurality of modified nucleotide molecules each comprising a 3′ blocking group, and a modified polymerase of claim 1.


SEQUENCE LISTING (7 sequences)

SEQ ID NO: 1 - protein, 783 aa, synthetic construct
  source 1..783: note = 9HY46 mut. 1 (D165A, E167A, L436I, Y437A, P438V, A515L)
MLFYPIDIDDSSEGILLFGITEDGRREKFIDSSYRPYFLVLPSDKKKAAAEIEKIVREKSLPVTEITEDKRIFYGQEREFIKIYTKRHQDLQKVRDAIKILEAKRGGSGSIIDEFEYQISPYRMYLAEKQLSCLQWLEIETHRNQIKSIKKATANPPKLKILAFAMAVIEVQRGTPSIVMISIFGEGLSRVITYQEAKYTIDATVVKNEKELIKNFIENVRDYDPDIIVGYNTDLYDMPILRERAKQLKVDIGILSRDNSGITLSKRARFTTARLIGRVHIDIFNFVFNILSPILQSEQLTLKNVSQELLGDTKLEMEYEEILQAWDKGHELDKLARYCLKDSELVYELSKMLLPDIMELTKITGQSLFDTSRMPYSQLVEWYYIKKAKQQNRVIPNQPKFDEIKERQKITYEGGFVKEPEVGLHKGIAVVDFASYAVSIIATYNISIDTLNCNCCKESGYKVPDFPYWFCKNIRGFESQAIEELLMERLELKKQLKTLNPESHEYLELDTKQKLLKTIINASYGYYAYPASRWYSKECAEAITALGRYWIKEVLTKAEQKGFHPIYADTDSAFLKIDSKEEVLQFIKEINQSLPGVMRLDFEDFYVKGLFVPRQIGGVAKKRYALVDEKGRLKIRGLEVVRRDLCRYARATQQEILRICLIEEDIPKAFKYLDERIKALREGKYDLKDLVVYEQLSKPVSEYKLISPHVAAAKRLLEKEIPVGEGSVIGYIIQKGSGSISERAYPIELAEPSKIDIDYYIENQILPTAMRILKVFKKENKLF

SEQ ID NO: 2 - protein, 783 aa, synthetic construct
  source 1..783: note = 9HY46 mut. 2 (D165A, E167A, L436I, Y437A, A515E, Y412F, V417I, V612H, I706K, V710M)
MLFYPIDIDDSSEGILLFGITEDGRREKFIDSSYRPYFLVLPSDKKKAAAEIEKIVREKSLPVTEITEDKRIFYGQEREFIKIYTKRHQDLQKVRDAIKILEAKRGGSGSIIDEFEYQISPYRMYLAEKQLSCLQWLEIETHRNQIKSIKKATANPPKLKILAFAMAVIEVQRGTPSIVMISIFGEGLSRVITYQEAKYTIDATVVKNEKELIKNFIENVRDYDPDIIVGYNTDLYDMPILRERAKQLKVDIGILSRDNSGITLSKRARFTTARLIGRVHIDIFNFVFNILSPILQSEQLTLKNVSQELLGDTKLEMEYEEILQAWDKGHELDKLARYCLKDSELVYELSKMLLPDIMELTKITGQSLFDTSRMPYSQLVEWYYIKKAKQQNRVIPNQPKFDEIKERQKITFEGGFIKEPEVGLHKGIAVVDFASIAPSIIATYNISIDTLNCNCCKESGYKVPDFPYWFCKNIRGFESQAIEELLMERLELKKQLKTLNPESHEYLELDTKQKELKTIINASYGYYAYPASRWYSKECAEAITALGRYWIKEVLTKAEQKGFHPIYADTDSAFLKIDSKEEVLQFIKEINQSLPGVMRLDFEDFYVKGLFHPRQIGGVAKKRYALVDEKGRLKIRGLEVVRRDLCRYARATQQEILRICLIEEDIPKAFKYLDERIKALREGKYDLKDLVVYEQLSKPVSEYKLKSPHMAAAKRLLEKEIPVGEGSVIGYIIQKGSGSISERAYPIELAEPSKIDIDYYIENQILPTAMRILKVFKKENKLF

SEQ ID NO: 3 - protein, 783 aa, synthetic construct
  source 1..783: note = 9HY46 WT
MLFYPIDIDDSSEGILLFGITEDGRREKFIDSSYRPYFLVLPSDKKKAAAEIEKIVREKSLPVTEITEDKRIFYGQEREFIKIYTKRHQDLQKVRDAIKILEAKRGGSGSIIDEFEYQISPYRMYLAEKQLSCLQWLEIETHRNQIKSIKKATANPPKLKILAFDMEVIEVQRGTPSIVMISIFGEGLSRVITYQEAKYTIDATVVKNEKELIKNFIENVRDYDPDIIVGYNTDLYDMPILRERAKQLKVDIGILSRDNSGITLSKRARFTTARLIGRVHIDIFNFVFNILSPILQSEQLTLKNVSQELLGDTKLEMEYEEILQAWDKGHELDKLARYCLKDSELVYELSKMLLPDIMELTKITGQSLFDTSRMPYSQLVEWYYIKKAKQQNRVIPNQPKFDEIKERQKITYEGGFVKEPEVGLHKGIAVVDFASLYPSIIATYNISIDTLNCNCCKESGYKVPDFPYWFCKNIRGFESQAIEELLMERLELKKQLKTLNPESHEYLELDTKQKALKTIINASYGYYAYPASRWYSKECAEAITALGRYWIKEVLTKAEQKGFHPIYADTDSAFLKIDSKEEVLQFIKEINQSLPGVMRLDFEDFYVKGLFVPRQIGGVAKKRYALVDEKGRLKIRGLEVVRRDLCRYARATQQEILRICLIEEDIPKAFKYLDERIKALREGKYDLKDLVVYEQLSKPVSEYKLISPHVAAAKRLLEKEIPVGEGSVIGYIIQKGSGSISERAYPIELAEPSKIDIDYYIENQILPTAMRILKVFKKENKLF

SEQ ID NO: 4 - protein, 794 aa, synthetic construct
  source 1..794: note = RX72 mut. (D184A, E186A, M439Y, Y440A, P441V, A521L)
MVAGEVRELEFWILDASYDVVKGHPVIYLWGITESGERIVVAEKNFRPYFYLVPENAISDALVERVRTALHTFKVLSVNAIERKLFGRTLNVLKVTITDPRDVPKAREVAAKIPGIRDVLEADIRFYMRYMVDTGISPSSWVLAKVRSAPVPNGWRVDEFFELVEAPRSLDKKSTPRLKTYAFAIACYNRYGEPDPERDPVLVISRTGEDGTVIFSQDEGSSEKKLLEEFVNDLVQYDPDVILGYNSNRFDWPYLLQRARVNGVKLSIGRNLGEPSQSVYGHFSVVGRANVDLYDYASELQEVKVKTLENVSEFLGVMKKSERVLIDTNKVYEYWDSKDKRSLLLRYAGDDARSTYLLGQVVLPFGIQLSSLVGLPLDQVFAASVGNRVEWFLIRQAFVFNELVPNSRERGEETYKGAIVLKPKPGVHKKIAVLDFSSYAVNIMIKYNISPDTYVPPEEHVDPSEVWVAPEVGHRFRKHPPGFYRKVLESLLEARRRLREKMKTLDPRSEEYRIYDERQKLIKVITNATYGYSGWSMARWYKREVAEATTAWGRELIKATIKKAQSLGLSIIYGDTDSIFVQFDEEKIGRLVEYVEKELGFEIKLDKVYEKVFFTESKKKYCGLLADGRVDLVGFEAVRGDWAEISKEIQERVVEIVLKEEDPWKAVDFVRKTITELSEGRIPLEKLVIWKTLSKDLDEYEVDAPHVVVARELKRLGYRVGKGAKIGYIVVKGAGKVSEKAKPLISVKSPDEVDTDYYVKRQIVPAALRILEYFGVKEEHFSTGRRQATLSDFF

SEQ ID NO: 5 - protein, 794 aa, synthetic construct
  source 1..794: note = RX72 WT
MVAGEVRELEFWILDASYDVVKGHPVIYLWGITESGERIVVAEKNFRPYFYLVPENAISDALVERVRTALHTFKVLSVNAIERKLFGRTLNVLKVTITDPRDVPKAREVAAKIPGIRDVLEADIRFYMRYMVDTGISPSSWVLAKVRSAPVPNGWRVDEFFELVEAPRSLDKKSTPRLKTYAFDIECYNRYGEPDPERDPVLVISRTGEDGTVIFSQDEGSSEKKLLEEFVNDLVQYDPDVILGYNSNRFDWPYLLQRARVNGVKLSIGRNLGEPSQSVYGHFSVVGRANVDLYDYASELQEVKVKTLENVSEFLGVMKKSERVLIDTNKVYEYWDSKDKRSLLLRYAGDDARSTYLLGQVVLPFGIQLSSLVGLPLDQVFAASVGNRVEWFLIRQAFVFNELVPNSRERGEETYKGAIVLKPKPGVHKKIAVLDFSSMYPNIMIKYNISPDTYVPPEEHVDPSEVWVAPEVGHRFRKHPPGFYRKVLESLLEARRRLREKMKTLDPRSEEYRIYDERQKAIKVITNATYGYSGWSMARWYKREVAEATTAWGRELIKATIKKAQSLGLSIIYGDTDSIFVQFDEEKIGRLVEYVEKELGFEIKLDKVYEKVFFTESKKKYCGLLADGRVDLVGFEAVRGDWAEISKEIQERVVEIVLKEEDPWKAVDFVRKTITELSEGRIPLEKLVIWKTLSKDLDEYEVDAPHVVVARELKRLGYRVGKGAKIGYIVVKGAGKVSEKAKPLISVKSPDEVDTDYYVKRQIVPAALRILEYFGVKEEHFSTGRRQATLSDFF

SEQ ID NO: 6 - protein, 781 aa, synthetic construct
  source 1..781: note = CR43 mut. (S178A, Q180A, M433Y, Y434A, P435V, A509L)
MIKDFFILDFSYDVIDNIPVIFIWAIDTEGQRIVLLEKSFRPYFYVVVDEKEVEYIIEEIKKLSKSGSPITNIIPIKGKKLFGQPVTVLRIETLIPAYVRIYRDEVLKVKGVKQVLEADIRFYMRYSIDKYIKPFHWIKAEVEEVENINFRVNKVYEIKNIKEQYVGSIPKLRKLSFAIAVYSKQGSPNPRKDPIVVIAVKTDDSAKQFVASDSDDKQIIKEFVAFFQEYDPDIVYGFNTNSFDLQYLLERAHSKGLKLDIGRRVNGEPSQGVYGHYSMTGRLNVDLAGFVNSVSEVKIKTLENICDYLGIVKKEDRVNLEWYEIPKMWEDKDKKNKVIQYVADNARSVYLLGESFLPFAIELTRITGLPLDQLSMASVGYRVEWLLMRESNKYNELIPNKEERTGESYEGGLVISPIQGIHENVIVLDFSSYAVSIMIKYNVGPDTLVKGECNDCWVSPIGHKFRKEPPGLYKFVLENLVKERREIKSKLKETKDEYEKRLLDERQKLLKVMANAFYGYMGWINARWYSREGAEAVTAWGRSIISDAAKIATEHGFKVIYGDTDSIFIKGNGDINKLQEIISQKFGIEIKMDKVYKRVLFTESKKRYAGITEDGRIDIVGFEAIRGDWCELAREVQRNVIEIILRTGKVDDAVRYVRKTILSLRRGEFKIEDLIIWKGLDKDVNEYAITAPHVVAAKKAIKAGYLVTKGSKIGYVIVKGTNKLSERAEPYFLVKDNSRIDISYYVDNQIIPSAMHVLKVFGVKEDSLKSGGTDILSFFKK

SEQ ID NO: 7 - protein, 781 aa, synthetic construct
  source 1..781: note = CR43 WT
MIKDFFILDFSYDVIDNIPVIFIWAIDTEGQRIVLLEKSFRPYFYVVVDEKEVEYIIEEIKKLSKSGSPITNIIPIKGKKLFGQPVTVLRIETLIPAYVRIYRDEVLKVKGVKQVLEADIRFYMRYSIDKYIKPFHWIKAEVEEVENINFRVNKVYEIKNIKEQYVGSIPKLRKLSFSIQVYSKQGSPNPRKDPIVVIAVKTDDSAKQFVASDSDDKQIIKEFVAFFQEYDPDIVYGFNTNSFDLQYLLERAHSKGLKLDIGRRVNGEPSQGVYGHYSMTGRLNVDLAGFVNSVSEVKIKTLENICDYLGIVKKEDRVNLEWYEIPKMWEDKDKKNKVIQYVADNARSVYLLGESFLPFAIELTRITGLPLDQLSMASVGYRVEWLLMRESNKYNELIPNKEERTGESYEGGLVISPIQGIHENVIVLDFSSMYPSIMIKYNVGPDTLVKGECNDCWVSPIGHKFRKEPPGLYKFVLENLVKERREIKSKLKETKDEYEKRLLDERQKALKVMANAFYGYMGWINARWYSREGAEAVTAWGRSIISDAAKIATEHGFKVIYGDTDSIFIKGNGDINKLQEIISQKFGIEIKMDKVYKRVLFTESKKRYAGITEDGRIDIVGFEAIRGDWCELAREVQRNVIEIILRTGKVDDAVRYVRKTILSLRRGEFKIEDLIIWKGLDKDVNEYAITAPHVVAAKKAIKAGYLVTKGSKIGYVIVKGTNKLSERAEPYFLVKDNSRIDISYYVDNQIIPSAMHVLKVFGVKEDSLKSGGTDILSFFKK